(19) United States
(12) Patent Application Publication
(10) Pub. No.: US 2025/0243467 A1
(43) Pub. Date: Jul. 31, 2025

(54) BONE MARROW ORGANOIDS PRODUCED FROM INDUCED PLURIPOTENT STEM CELLS AND USES OF THESE ORGANOIDS

(71) Applicant: PRISM INGENUITY GMBH, MÜNCHEN (DE)

(72) Inventors: Stephanie Caroline FRENZ-WIESSNER, MÜNCHEN (DE); Christoph KLEIN, MÜNCHEN (DE); Isabel Hülya GÖK, MÜNCHEN (DE)

(21) Appl. No.: 18/854,608

(22) PCT Filed: Apr. 4, 2023
(86) PCT No.: PCT/EP2023/058824

§ 371 Date: Oct. 7, 2024

(30) Foreign Application Priority Data
Apr. 8, 2022 (LU) LU501820

(51) Int. Cl. C12N 5/071; A61K 35/28; A61K 35/44

(52) U.S. Cl. CPC C12N 5/0697; A61K 35/28; A61K 35/44; C12N 5/0691; C12N 2501/115; C12N 2501/125; C12N 2501/145; C12N 2501/15; C12N 2501/155; C12N 2501/16; C12N 2501/165; C12N 2501/2303; C12N 2501/26; C12N 2501/727; C12N 2503/02; C12N 2503/04; C12N 2506/02; C12N 2506/45; C12N 2513/00

(57) ABSTRACT

The present invention relates to a method for producing mammalian vascular networks or mature mammalian bone marrow organoids. Furthermore provided are uses of the vascular network or mammalian bone marrow organoids as produced for use in the treatment of bone marrow related diseases, for the in-vitro production of BMOs or mammalian blood cells, as a model system in the pathogenesis of a bone marrow related disease, and as a system for identifying and/or testing pharmaceutically effective compounds for treating or preventing of a bone marrow related disease.

The present invention relates to a method for producing mammalian vascular networks or mature mammalian bone marrow organoids. Furthermore, provided are uses of the vascular network or mammalian bone marrow organoids as produced for use in the treatment of bone marrow related diseases, for the in-vitro production of BMOs or mammalian blood cells, as a model system in the pathogenesis of a bone marrow related disease, and as a system for identifying and/or testing pharmaceutically effective compounds for treating or preventing of a bone marrow related disease.

### BACKGROUND OF THE INVENTION

Human postnatal hematopoiesis takes place in the bone marrow and involves a strictly regulated process of constant differentiation of hematopoietic stem cells (HSC) into mature blood cells while maintaining an HSC pool through self-renewal. The surrounding microenvironment is called the bone marrow niche and consists of a heterogeneous cell population, including mesenchymal cells (e.g. pericytes, adipocytes) and endothelial cells. The niche of the bone marrow plays an important role in regulating and maintaining hematopoiesis throughout the whole life (1).

A dense vascular network within the bone marrow is essential because it supplies other niche cells with nutrients, growth factors, and critical cell-cell-interactions. In addition, endothelial cells covered by perivascular PDGFRβ+ mesenchymal cells (pericytes) directly promote hematopoietic homeostasis through the secretion of various factors; therefore, the HSCs are often located in the immediate vicinity of the blood vessels (2).

Impaired hematopoiesis can be caused by specific genetic mutations and manifests itself in the case of germline mutations in the form of congenital bone marrow failure (IBMFS), such as, for example, severe congenital neutropenia, or in somatic mutations in the form of diseases such as myelodysplasia and leukemia. Interestingly, disruption of the bone marrow microenvironment has been shown to trigger myelodysplasia (3).

Human organoid models have been established in recent years as a model system for studying the development of diseases of various tissues. Organoids are self-organized 3D structures that mimic the most important functions and structural organization of organs and can be differentiated from induced pluripotent stem cells (iPSC). Organoids are partly superior to conventional 2D cultures, because they mimic the natural environment of certain cells through cell-cell interactions as well as cell-matrix interactions (7, Hofer, M., Lutolf, M. P. Engineering organoids. Nat Rev Mater 6, 402-420 (2021). https://doi.org/10.1038/s41578-021-00279-y).

Organoid formation and maturation are preceded by a single cell or small cell-cluster expansion and reorganization. There are two main types of organoids based upon the choice of stem cells. The first is derived from PSCs that include both embryonic stem cells (ESCs) and iPSCs and the second type is derived from organ-specific adult stem cells (ASCs). A variety of workflows have been developed to generate organoids; however, specialized organoid types require unique culture methods, and not all general workflows are appropriate. The choices of cell culture conditions and the 3D matrix are critical for this complex organization.

WO2019122388A1 discloses co-cultures of organoids and immune cells, and methods of using these to identify agents for treating diseases.

Breunig et al. (Differentiation of human pluripotent stem cells into pancreatic duct-like organoids. STAR Protoc. 2021 Dec. 8; 2(4):100913. doi: 10.1016/j.xpro.2021.100913. PMID: 34917972; PMCID: PMC8669107) describe a scalable in vitro differentiation protocol to guide human pluripotent stem cells stepwise into pancreatic duct-like organoids. The protocol mimics pancreatic duct development and was successfully used to model the onset and progression of pancreatic ductal adenocarcinoma; the approach is suitable for multiple downstream applications. However, the protocol is cost- and time-intensive.

Vallmajo-Martin, Q., et al. (in: PEG/HA Hybrid Hydrogels for Biologically and Mechanically Tailorable Bone Marrow Organoids. Adv. Funct. Mater. 2020, 30, 1910282. https://doi.org/10.1002/adfm.201910282) disclose that bone marrow (BM) organoids provide powerful tools to study the vital interplay between the BM microenvironment and resident cells. A transglutaminase (TG) crosslinked system that seamlessly incorporates poly(ethylene glycol) (PEG) and hyaluronic acid (HA) into hybrid hydrogels for the formation of BM analogues is presented. Utility of the TG-PEG/HA hybrid hydrogels to maintain, expand, or differentiate human bone marrow-derived stromal cells and human hematopoietic stem and progenitor cells in vitro is demonstrated. TG-PEG/HA hybrid hydrogels are described as superior to currently used natural biomaterials in forming humanized BM organoids in a xenograft model. The engineered humanized BM organoids as presented may be effective tools for the study of this intricate organ.

Isern J, et al. (in Self-renewing human bone marrow mesenspheres promote hematopoietic stem cell expansion. Cell Rep. 2013 May 30; 3(5):1714-24. doi: 10.1016/j.celrep.2013.03.041. Epub 2013 Apr. 25. PMID: 23623496) discuss strategies for expanding hematopoietic stem cells (HSCs) include coculture with cells that recapitulate their natural microenvironment, such as bone marrow stromal stem/progenitor cells (BMSCs). Plastic-adherent BMSCs may be insufficient to preserve primitive HSCs. They describe a method of isolating and culturing human BMSCs as nonadherent mesenchymal spheres. Human mesenspheres were derived from CD45-CD31− CD71− CD146+ CD105+ nestin+ cells but could also be simply grown from fetal and adult BM CD45−-enriched cells. Human mesenspheres robustly differentiated into mesenchymal lineages. In culture conditions where they displayed a relatively undifferentiated phenotype, with decreased adherence to plastic and increased self-renewal, they promoted enhanced expansion of cord blood CD34+ cells through secreted soluble factors. Expanded HSCs were serially transplantable in immunodeficient mice and significantly increased long-term human hematopoietic engraftment. They discuss the way for culture techniques that preserve the self-renewal of human BMSCs and their ability to support functional HSCs.

Sun et al. (in: Generation of vascularized brain organoids to study neurovascular interactions. Elife. 2022 May 4; 11:e76707. doi: 10.7554/eLife.76707. PMID: 35506651; PMCID: PMC9246368) induced vessel and brain organoids, respectively, and then fused two types of organoids together to obtain vascularized brain organoids.

Janagama and Hui (in: 3-D Cell Culture Systems in Bone Marrow Tissue and Organoid Engineering, and BM Phantoms as In Vitro Models of Hematological Cancer Therapeutics-A Review. Materials (Basel). 2020; 13(24):5609. Published 2020 Dec. 9. doi:10.3390/ma13245609) review the state-of-the-art in bone and marrow tissue engineering (BMTE) and hematological cancer tissue engineering (HCTE) in light of the recent interest in bone marrow environment and pathophysiology of hematological cancers. They focus on engineered BM tissue and organoids as in vitro models of hematological cancer therapeutics, along with identification of BM components and their integration as synthetically engineered BM mimetic scaffolds. In addition, the review details interaction dynamics of various BM and hematologic cancer (HC) cell types in co-culture systems of engineered BM tissues/phantoms as well as their relation to drug resistance and cytotoxicity. Interaction between hematological cancer cells and their niche, and the difference with respect to the healthy niche microenvironment narrated. Future perspectives of BMTE for in vitro disease models, BM regeneration and large-scale ex vivo expansion of hematopoietic and mesenchymal stem cells for transplantation and therapy are explained. They conclude by overviewing the clinical application of biomaterials in BM and HC pathophysiology and its challenges and opportunities.

Bessy T (in: Bioengineering the Bone Marrow Vascular Niche. Front Cell Dev Biol. 2021; 9:645496. Published 2021 Apr. 28. doi:10.3389/fcell.2021.645496) provide another comprehensive review with a focus on engineering vascularized BM niche models, and summarize current approaches including bioengineered microfluidic chips.

Cornelia Lee-Thedieck, et al. (in: The extracellular matrix of hematopoietic stem cell niches, Advanced Drug Delivery Reviews, Volume 181, 2022, 114069, https://doi.org/10.1016/j.addr.2021.114069) review that hematopoietic stem cells (HSCs) are the life-long source of all types of blood cells. Their function is controlled by their direct microenvironment, the HSC niche in the bone marrow. Although the importance of the extracellular matrix (ECM) in the niche by orchestrating niche architecture and cellular function is widely acknowledged, it is still underexplored. In the review, they provide a comprehensive overview of the ECM in HSC niches. For this purpose, they briefly outline HSC niche biology and then review the role of the different classes of ECM molecules in the niche one by one and how they are perceived by cells. Matrix remodeling and the emerging importance of biophysics in HSC niche function are discussed. Finally, the application of the current knowledge of ECM in the niche in form of artificial HSC niches for HSC expansion or targeted differentiation as well as drug testing is reviewed.

Since mouse models often do not fully recapitulate the human phenotype due to differences in hematopoiesis between mice and humans, an alternative approach is needed to investigate the genetic causes and mechanisms that lead to bone marrow disease. Previous studies on in vitro modelling of the niche in the human bone marrow rely on the use of primary endothelial cells and mesenchymal cells, but these do not reflect the multicellular complexity of the natural niche system, and broader applications are limited by the supply and limited lifespan of these cells (4-6).

As seen from the above, there is an unmet need for new in vitro approaches that replicate hematopoiesis in a complex bone marrow-like niche system to study hematopoietic diseases and develop new therapies. It is therefore an object of the present invention, to provide respective approaches and methods that are used to establish suitable systems in order to study hematopoietic diseases and develop new therapies. Other objects and advantages will become apparent to the person of skill upon studying the present description of the invention at hand.

In a first aspect thereof, the present invention solves the above problem by providing a method for producing mature mammalian bone marrow organoids, comprising the steps of a) generating embryoid bodies from substantially single induced pluripotent stem cells (iPSCs) obtained from at least one mammal, comprising culturing single iPSCs in an aggregation medium in the presence of at least one Rho-associated protein kinase (ROCK)-inhibitor for about 1 day, followed by culturing the embryoid bodies in a suitable serum-free, stabilized cell culture medium suitable for the feeder-free maintenance and expansion of human embryonic stem cells without ROCK-inhibitor for about 48 hours; b) inducing mesoderm in said embryoid bodies as formed in step a), comprising culturing said embryoid bodies in a suitable serum-free, stabilized cell culture medium suitable for the feeder-free maintenance and expansion of human embryonic stem cells, wherein the medium is supplemented with 80 ng/ml Bone morphogenetic protein 4 (BMP4), 4 μM of glycogen synthase kinase (GSK) 3 inhibitor, and 80 ng/ml Vascular Endothelial Growth Factor (VEGF), for about 48 hours, with resuspension of the mesoderm-induced embryoid bodies at about every 24 hours; c) replacing the medium of the culture of b) with Essential 6-medium, supplemented with 80 ng/ml VEGF, 25 ng/ml fibroblast growth factor (FGF)-2, 50 ng/ml stem cell factor (SCF), and 2 μM SB431542 for about 48 hours, with resuspension of the embryoid bodies at about every 24 hours and gentle shaking; d) embedding of the mesoderm-induced embryoid bodies of step c) into a suitable polymerized 3D collagen I/Matrigel® matrix followed by overlaying the matrix with StemPro®-34 medium supplemented with 80 ng/ml VEGF, 25 ng/ml FGF-2, 50 ng/ml SCF and 2 μM SB431542 for about 48 hours, followed by cytokine replacement with 50 ng/mL VEGF, 50 ng/mL SCF, 50 ng/mL TL-3, 50 ng/mL Flt-3L, and 5 ng/mL TPO for about 48 hours, and a cytokine boost to 25 ng/mL VEGF, 50 ng/mL SCF, 50 ng/mL IL-3, 50 ng/mL Flt-3L, and 5 ng/mL TPO for about 48 hours; e) extracting individual vascular networks that have been generated in step d) followed by culturing in StemPro®-34 medium, supplemented with 25 ng/ml VEGF, 50 ng/ml SCF, 50 ng/ml IL-3, 50 ng/ml Flt-3L and 5 ng/ml TPO, with refreshing the medium every 3 to 4 days for about 7 to 11 days, whereby the mature mammalian bone marrow organoids are produced wherein preferably no lineage-directing cytokines, such as EPO, TL-6 and/or G-CSF, are used in the method. Preferably, the mammalian bone marrow organoids are mature organoids, i.e., they show the physiological main characteristics of the respective tissue(s) in vivo.

In a second aspect thereof, the present invention solves the above problem by providing a vascular network or mammalian bone marrow organoid, preferably a mature organoid, produced according to the method according to the present invention, or a pharmaceutical composition comprising the vascular network and/or mature mammalian bone marrow organoid according to the present invention. Furthermore provided are the vascular network or mammalian bone marrow organoid, preferably a mature organoid, produced according to the method according to the present invention, or a pharmaceutical composition comprising the vascular network and/or mature mammalian bone marrow organoid according to the present invention for use in the treatment of diseases.

In a third aspect thereof, the present invention solves the above problem by providing the use of the vascular network or mammalian bone marrow organoid or the pharmaceutical composition according to the present invention as a model system in the pathogenesis of a bone marrow related disease, such as, for example, a hematological disease, severe congenital neutropenia, myelofibrosis, blood cell cancers, anemia, thrombocytopenia, inborn errors of hematopoiesis and immunity, conditions related to HIV, sickle cell disease, and complications from chemotherapy or transfusions.

In a fourth aspect thereof, the present invention solves the above problem by providing the use of the vascular network or mammalian bone marrow organoid or the pharmaceutical composition according to the present invention as a model system for identifying and/or testing pharmaceutically effective compounds for treating or preventing of a bone marrow related disease, such as, for example, a hematological disease, severe congenital neutropenia, myelofibrosis, blood cell cancers, anemia, thrombocytopenia, inborn errors of hematopoiesis and immunity, conditions related to HIV, sickle cell disease, and complications from chemotherapy or transfusions. Another aspect of this embodiment is a method for screening a pharmaceutically effective compound for treating or preventing of a bone marrow related disease, such as, for example, a hematological disease, severe congenital neutropenia, myelofibrosis, blood cell cancers, anemia, thrombocytopenia, inborn errors of hematopoiesis and immunity, conditions related to HIV, sickle cell disease, and complications from chemotherapy or transfusions, comprising the use of the vascular network or mammalian bone marrow organoid or the pharmaceutical composition according to the present invention as a model system.

In a fifth aspect thereof, the present invention solves the above problem by providing an assembloid, comprising the vascular network or mature mammalian bone marrow organoid according to the present invention with at least one additional iPSC-derived organoid.

In a sixth aspect thereof, the present invention solves the above problem by providing the use of the vascular network or mammalian bone marrow organoid or the pharmaceutical composition according to the present invention for the in-vitro production of BMOs or mammalian blood cells, in particular autologous BMOs or mammalian blood cells, in particular for transplantation purposes.

In a seventh aspect thereof, the present invention solves the above problem by providing the pharmaceutically effective amount of the vascular network or mature mammalian bone marrow organoid or the assembloid or the pharmaceutical composition according to the present invention for use in the treatment of a bone marrow related disease, such as, for example, a hematological disease, severe congenital neutropenia, myelofibrosis, blood cell cancers, anemia, thrombocytopenia, inborn errors of hematopoiesis and immunity, conditions related to HIV, sickle cell disease, and complications from chemotherapy or transfusions, preferably for transplantation. Another aspect of this embodiment is a method for treating a bone marrow related disease, such as, for example, a hematological disease, severe congenital neutropenia, myelofibrosis, blood cell cancers, anemia, thrombocytopenia, inborn errors of hematopoiesis and immunity, conditions related to HIV, sickle cell disease, and complications from chemotherapy or transfusions, comprising administering to a subject in need thereof a pharmaceutically effective amount of the vascular network or mammalian bone marrow organoid or the pharmaceutical composition according to the present invention. Preferably, the vascular network or mammalian bone marrow organoid or the assembloid or the pharmaceutical composition according to the present invention is administered as transplant, e.g., an autologous transplant. Another aspect of this embodiment is a method for transplanting the vascular network or mammalian bone marrow organoid or the pharmaceutical composition according to the present invention into a mammalian subject in need thereof, preferably in order to treat a bone marrow related disease, such as, for example, a hematological disease, severe congenital neutropenia, myelofibrosis, blood cell cancers, anemia, thrombocytopenia, inborn errors of hematopoiesis and immunity, conditions related to HIV, sickle cell disease, and/or complications from chemotherapy or transfusions.

The inventors present a novel approach to generate bone marrow organoids (BMOs) (the term shall wherever possible include both bone marrow organoids and mature bone marrow organoids as produced) that mimic important structural and cellular features of the human bone marrow niche. These BMOs are generated exclusively from human induced pluripotent stem cells (hiPSCs) and have a hematopoietic, stromal (mesenchymal) and vascular compartment. The inventors show that this system induces the formation of mature blood cells of the myeloid lineage, such as neutrophil, eosinophil and basophil granulocytes and monocytes, as well as megakaryocytic lineage, mast cells, dendritic cells, lymphoid progenitor cells and early erythroid progenitor cells. Furthermore, the inventors can show that the stromal compartment consists of mesenchymal stem cells and progenitor cells, such as CXCL12-rich reticular (CAR) cells and perivascular cells, while the vascular compartment consists of self-assembling, connected and lumen-forming endothelial cells.

Khan et al. (in: Human Bone Marrow Organoids for Disease Modeling, Discovery, and Validation of Therapeutic Targets in Hematologic Malignancies. Cancer Discov. 2023 Feb. 6; 13(2):364-385. doi: 10.1158/2159-8290.CD-22-0199. PMID: 36351055; PMCID: PMC9900323) describe a step-wise, directed-differentiation protocol in which organoids are generated from iPSCs committed to mesenchymal, endothelial and hematopoietic lineages. These 3-dimensional structures were reported to capture key features of human bone marrow—stroma, lumen-forming sinusoidal vessels and myeloid cells including pro-platelet forming megakaryocytes. The organoids were reported to support the engraftment and survival of cells from patients with blood malignancies, including cancer types notoriously difficult to maintain ex vivo. Fibrosis of the organoid occurred following TGFβ stimulation and engraftment with myelofibrosis but not healthy donor-derived cells. Khan et al. do not mention exact concentrations as used in their protocol.

Importantly, no Wnt-activator (e.g. CHIR99021) or Nodal-Inhibitor (e.g. SB431542) was used, which has been shown to be important for mesodermal patterning leading to induction of definitive hematopoiesis. Generation of primitive progenitors (KDR⁺CD235a⁺) depends on stage-specific Activin-nodal signaling and inhibition of the Wnt-β-catenin pathway, whereas specification of definitive progenitors (KDR⁺CD235a⁻) requires Wnt-β-catenin signaling during this same time frame. (Sturgeon, C. M., Ditadi, A., Awong, G., Kennedy, M. & Keller, G. Wnt signaling controls the specification of definitive and primitive hematopoiesis from human pluripotent stem cells. Nat. Biotechnol. 32, 554-561 (2014)):

Also, lineage-directed cytokines, such as EPO, IL-6 and G-CSF were used in Khan et al., which prevents the possibility to study intrinsic cytokine signaling within the bone marrow organoids, and likely presents an obstacle for modelling of genetic bone marrow failure syndromes, due to some possible rescue effect of these cytokines.

In the present invention maturation of neutrophil granulocytes within the BMOs, proceeding without addition of recombinant cytokines, is reminiscent of the in vivo situation (see below).

Therefore, structurally, the present invention differs from Khan et al. in that, for example, the blood vessels consist of arterial-type endothelial cells instead of sinusoids. This is crucial, since long-term HSCs have been shown to arise from arterial type hemogenic endothelium (Calvanese et al., Nature 2022). For the mesenchymal cell compartment, a desirable heterogeneous mesenchymal cell type composition including mesenchymal stem and progenitor cells giving rise to osteogenic, chondrogenic and adipogenic precursor cells was found. For the hematopoietic cells clear evidence by scRNA-sequencing of potential differentiation into all hematopoietic lineages was found, instead of only erythromyeloid in Khan et al. Finally, no transplantation was performed by Khan et al., in contrast to the present organoids that were transplantable into NOD-SCID mice in vivo and showed engraftment of human CD45+ in murine bone marrow indicating HSC properties of BMO-derived cells.

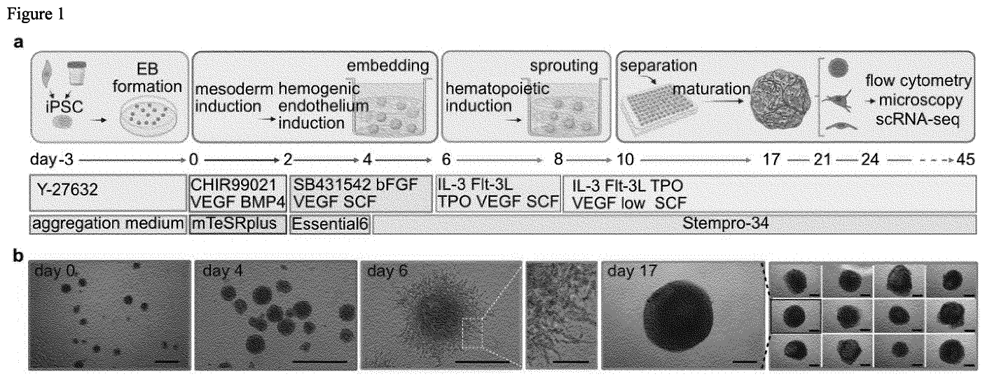

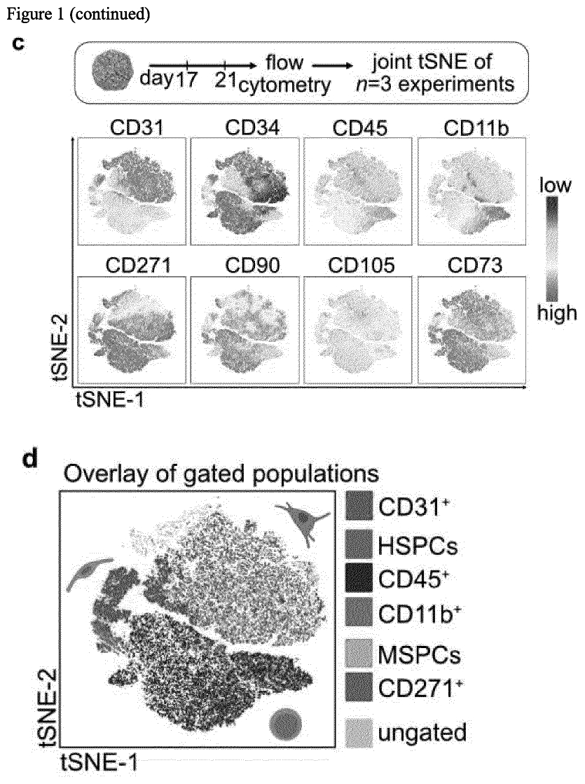

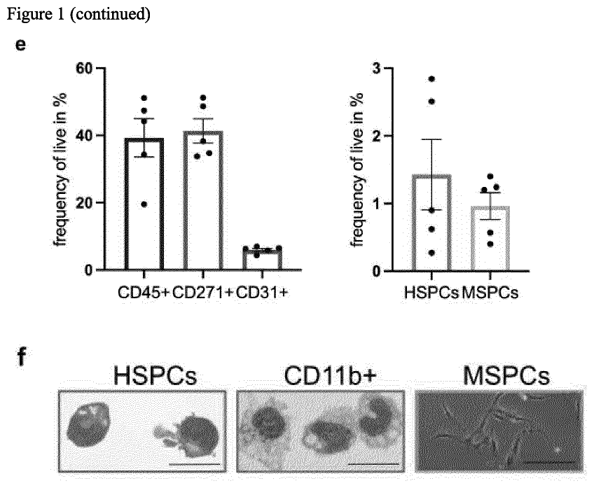

The organoids according to the present invention showed a consistent spheroidal morphology (see FIG. 1) across differentiations. This could also be reproduced with a different iPS cell line that was derived from renal-epithelial cells.

Therefore, the inventors developed an improved protocol that allows for a reliable production of mammalian bone marrow organoids, in particular mature mammalian bone marrow organoids derived solely from iPSCs and composed of blood cells and various niche cells such as endothelial cells and mesenchymal cells. The method according to the present invention comprises the steps of a) generating embryoid bodies from substantially single induced pluripotent stem cells (iPSCs) obtained from at least one mammal, comprising culturing single iPSCs in an aggregation medium in the presence of at least one Rho-associated protein kinase (ROCK)-inhibitor for about 1 day, followed by culturing the embryoid bodies in a suitable serum-free, stabilized cell culture medium suitable for the feeder-free maintenance and expansion of human embryonic stem cells without ROCK-inhibitor for about 48 hours; b) inducing mesoderm in said embryoid bodies as formed in step a), comprising culturing said embryoid bodies in a suitable serum-free, stabilized cell culture medium suitable for the feeder-free maintenance and expansion of human embryonic stem cells, wherein the medium is supplemented with 80 ng/ml Bone morphogenetic protein 4 (BMP4), 4 μM of glycogen synthase kinase (GSK) 3 inhibitor, and 80 ng/ml Vascular Endothelial Growth Factor (VEGF), for about 48 hours, with resuspension of the mesoderm-induced embryoid bodies at about every 24 hours; and gentle shaking c) replacing the medium of the culture of b) with Essential 6-medium, supplemented with 80 ng/ml VEGF, 25 ng/ml fibroblast growth factor (FGF)-2, 50 ng/ml stem cell factor (SCF), and 2 μM SB431542 for about 48 hours, with resuspension of the embryoid bodies at about every 24 hours; d) embedding of the mesoderm-induced embryoid bodies of step c) into a suitable polymerized 3D collagen I/Matrigel® matrix followed by overlaying the matrix with StemPro®-34 medium supplemented with 80 ng/ml VEGF, 25 ng/ml FGF-2, 50 ng/ml SCF and 2 μM SB431542 for about 48 hours, followed by cytokine replacement with 50 ng/mL VEGF, 50 ng/mL SCF, 50 ng/mL TL-3, 50 ng/mL Flt-3L, and 5 ng/mL TPO for about 48 hours, and a cytokine boost to 25 ng/mL VEGF, 50 ng/mL SCF, 50 ng/mL IL-3, 50 ng/mL Flt-3L, and 5 ng/mL TPO for about 48 hours; e) extracting individual vascular networks that have been generated in step d) followed by culturing in StemPro®-34 medium, supplemented with 25 ng/ml VEGF, 50 ng/ml SCF, 50 ng/ml TL-3, 50 ng/ml Flt-3L and 5 ng/ml TPO, with refreshing the medium every 3 to 4 days for about 7 to 11 days, whereby the mammalian bone marrow organoids are produced, wherein preferably no lineage-directing cytokines, such as EPO, TL-6 and/or G-CSF, are used. Preferred is the method for producing mammalian bone marrow organoids according to the present invention, further comprising the step of culturing the mature organoids for a maximum of about 47 days, preferably about 60 days.

The invention provides a vascularized BMO preparation or system derived exclusively from iPSCs (in particular hiPSCs), mimicking hematopoiesis in a multicellular context of native bone marrow, comprising blood cells and various bone marrow “niche cells”, such as endothelial cells and mesenchymal cells. The system allows the modeling of bone-marrow related diseases, i.e. the possibility of studying the interaction between niche cells and hematopoietic cells in the pathogenesis of hematological diseases. This can be furthermore achieved in the context of drug testing, i.e., studying the effects of drugs on the system, which allows the testing of new therapies in the inventive complex mammalian, e.g., human, in-vitro model system. Possible is also an implementation in a high-throughput format, for example in a 96-well-plate format. Furthermore, the “products” as generated can be used in therapeutic applications, i.e., bone-marrow related diseases, such as hematological diseases. The method provides the possibility of a transplantation of human BMOs as produced or the improved in vitro generation of human blood cells from iPSCs, e.g., autologous patient-specific production, and subsequent infusion or transplantation.

Until now, the ex-vivo expansion of HSCs for stem cell transplantations was not possible. Advantageously, the vascular network or mature mammalian bone marrow organoid according to the present invention maintain stemness of hematopoietic stem cells (HSC), making it possible to keep HSC in vitro for further quality control studies and to sort only those cells for therapeutic application that show desired molecular changes without any unwanted side effects.

Preferred is the method for producing mammalian bone marrow organoids according to the present invention, further comprising the step of fixing the vascular networks in step e) and performing immunofluorescence analysis and/or dissociating the vascular networks into individual cells and performing flow cytometry analysis and/or live-cell imaging. This can be done in order to characterize the components of the networks as developed, and/or to study any effects of drugs to be tested (see also below).

Preferred is the method for producing mammalian bone marrow organoids according to the present invention, further comprising the step of fixing the (mature) organoids as produced, and performing immunofluorescence analysis and/or dissociating the mature organoids into individual cells and performing flow cytometry analysis and/or live-cell imaging. This can be done in order to characterize the components of the organoids (e.g. the maturation) as developed, and/or to study any effects of drugs to be tested (see also below).

In the first step of the method according to the present invention, embryoid bodies are generated from substantially single/individual induced pluripotent stem cells (iPSCs) obtained from at least one mammal. Individual cells can be obtained by several ways, for example by mechanical dissociation (e.g., using a pipette or the like). Nevertheless, preferred is the method for producing mammalian bone marrow organoids according to the present invention, wherein the single/individual iPSCs are provided by dissociating iPSC cells into single cells with Accutase® digestion, which is a more gentle procedure. Accutase® is gentle on cells and auto-inhibits at 37° C. without the need for a neutralizing solution, like with trypsin. Accutase® is commercially available from Sigma, and works for all mammalian iPSCs. The method further comprises culturing individual/single iPSCs in an aggregation medium in the presence of at least one Rho-associated protein kinase (ROCK)-inhibitor for about 1 day.

Preferred is the method for producing mammalian bone marrow organoids according to the present invention, wherein the aggregation medium is KnockOut DMEM/F12 (Thermo Fisher) with 20% serum substitute, 1% L-glutamine, 1% non-essential amino acids, 1% penicillin-streptomycin, and 100 μM β-mercaptoethanol.

In the method for producing mammalian bone marrow organoids according to the present invention, in the first step, the cells are furthermore cultured in the presence of at least one Rho-associated protein kinase (ROCK)-inhibitor. ROCK inhibition enables maintenance of stem cell phenotype; its effects on metabolism are unknown.

Preferred is the method for producing mammalian bone marrow organoids according to the present invention, wherein the ROCK-inhibitor is selected from the group consisting of Y-27632 ((1R,4r)-4-((R)-1-aminoethyl)-N-(pyridin-4-yl)cyclohexanecarboxamide), fasudil, and a TS-f ROCK-inhibitor as disclosed in Shen et al. (Shen, M., Tian, S., Pan, P. et al. Discovery of Novel ROCK1 Inhibitors via Integrated Virtual Screening Strategy and Bioassays. Sci Rep 5, 16749 (2015). https://doi.org/10.1038/srep16749, herewith incorporated by reference), in particular TS-f5 or TS-f22. Other suitable ROCK-inhibitors are known to the person of skill and are disclosed in the respective literature.

Usually and preferably, step a) is performed for 60 to 84 hours, preferably for about 72 hours, i.e., about 3 days, as mentioned above, the medium is changed to a suitable medium (e.g., TeSRplus) without Rock-inhibitor after about 24 hours.

In the context of the present invention, the term “about” shall mean a deviation form a given value of +/−10%, unless indicated otherwise.

In the context of the present invention, culturing is generally done at about 37° C., unless indicated otherwise.

In the second step of the method according to the present invention, mesoderm is induced in said embryoid bodies as formed in step a). The step comprises culturing the embryoid bodies in a suitable serum-free, stabilized cell culture medium suitable for the feeder-free maintenance and expansion of human embryonic stem cells, and human iPSCs.

Preferably, the serum-free, stabilized cell culture medium suitable for the feeder-free maintenance and expansion of human embryonic stem cells is mTeSR® Plus medium (STEMCELL Technologies, Fisher Scientific). The medium is supplemented with 80 ng/ml Bone morphogenetic protein 4 (BMP4), 4 μM of glycogen synthase kinase (GSK) 3 inhibitor, and 80 ng/ml Vascular Endothelial Growth Factor (VEGF).

Inhibition of GSK3 e.g., by CHIR99021, promotes mESC self-renewal by stabilizing cytoplasmic β-catenin, an essential component of the canonical Wnt signaling pathway, abrogating TCF3-mediated transcriptional repression of pluripotency-associated genes including Oct4, Nanog, Tfcp2l1, and Esrrb. Therefore, preferred is the method for producing mammalian bone marrow organoids according to the present invention, wherein the glycogen synthase kinase (GSK) 3 inhibitor is CHIR99021 or NPD13432, an aurone derivative (Hiroki Kobayashi, et al. A novel GSK3 inhibitor that promotes self-renewal in mouse embryonic stem cells, Bioscience, Biotechnology, and Biochemistry, Volume 84, Issue 10, 2 Oct. 2020, Pages 2113-2120, https://doi.org/10.1080/09168451.2020.1789445). The glycogen synthase kinase (GSK) 3 inhibitor may be combined with a MEK inhibitor (e.g., PD0325901, called “2i”) which supports the long-term self-renewal of mouse embryonic stem cells (mESCs), while blockade of the MEK/ERK pathway by PD0325901 increases the expression of Nanog, Tfcp2l1, and Klf4 in mESCs, thereby promoting self-renewal.

Usually and preferably, step b) is performed for 36 to 60 hours, preferably for about 48 hours, i.e., about 2 days, with gentle mixing or resuspension of the mesoderm-induced embryoid bodies at about every 24 hours.

In the third step of the method according to the present invention, the medium of the culture of step b) is replaced with Essential 6-medium, supplemented with 80 ng/ml VEGF, 25 ng/ml fibroblast growth factor (FGF)-2, 50 ng/ml stem cell factor (SCF), and a suitable inhibitor of the TGF-β/Activin/NODAL pathway, preferably 2 μM SB431542 (STEMCELL Technologies). SB431542 is a selective and potent inhibitor of the TGF-β/Activin/NODAL pathway that inhibits ALK5 (IC₅₀=94 nM), ALK4 (IC₅₀=140 nM), and ALK7 by competing for the ATP binding site. It does not inhibit the BMP type I receptors ALK2, ALK3, and ALK6. Essential 6 Medium (Thermo Fisher) is a feeder-free and xeno-free medium that supports the reprogramming of somatic cells and the spontaneous or directed differentiation of human pluripotent stem cells (PSCs).

Usually and preferably, step c) is performed for 36 to 60 hours, preferably for about 48 hours, i.e., about 2 days, with gentle mixing or resuspension of the embryoid bodies at about every 24 hours and placing the embryoid bodies on rocking shaker.

The fourth step of the method according to the present invention comprises the embedding of the mesoderm-induced embryoid bodies as generated in step c). Embedding the embryoid bodies into a suitable polymerized 3D matrix prevents the embryoid bodies from sinking to the bottom of the dish or well, which would hinder or impart the further development into mature embryoid bodies and vascular structures. The extracellular matrix (ECM) further provides biochemical cues and structural support, such as porosity and stiffness which mediates signaling for cell migration, cell behavior and polarization in organoid structures. In general, any suitable polymerized 3D matrix may be used. The present invention preferably uses a collagen I/Matrigel® matrix bottom layer in the culturing vessel (e.g., a vial, dish or well). For example, 500 l/well of collagen I-Matrigel® mixture is first prepared and polymerized at 37° C. for about 1 hour as the lowest layer. Then, embryoid bodies were resuspended in 500 l/well of the same collagen-I-Matrigel mixture and a second layer was prepared, which was also polymerized at 37° C. for about 1 hour. The two layers were then overlayed with a suitable medium, such as, for example, StemPro®-34 medium, supplemented with 80 ng/ml VEGF, 25 ng/ml FGF-2, 50 ng/ml SCF and a suitable inhibitor of the TGF-β/Activin/NODAL pathway as above, preferably 2 μM SB431542 (STEMCELL Technologies). StemPro®-34 SFM (e.g., from Thermo Fisher) is a serum-free medium specifically formulated to support the development of human hematopoietic cells in culture (Burridge P W, et al. A universal system for highly efficient cardiac differentiation of human induced pluripotent stem cells that eliminates interline variability. PLoS One. 2011 Apr. 8; 6(4):e18293. doi: 10.1371/journal.pone.0018293. PMID: 21494607; PMCID: PMC3072973).

Usually and preferably, the first part of step d) is performed for 36 to 60 hours, preferably for about 48 hours, i.e., about 2 days. Then, a cytokine replacement followed in the same medium with 50 ng/mL VEGF, 50 ng/mL SCF, 50 ng/mL IL-3, 50 ng/mL Flt-3L, and 5 ng/mL TPO. Usually and preferably, the second part of step d) is performed for 36 to 60 hours, preferably for about 48 hours, i.e., about 2 days. Finally, a cytokine boost followed in the same medium to 25 ng/mL VEGF, 50 ng/mL SCF, 50 ng/mL IL-3, 50 ng/mL Flt-3L, and 5 ng/mL TPO, Usually and preferably, the third part of step d) is performed for 36 to 60 hours, preferably for about 48 hours, i.e., about 2 days. At the end of the fourth step of the method according to the present invention, vascular networks are generated. These also already contain hematopoietic cells, and are thus called “sprouting embryoid bodies”.

In the fifth step of the method according to the present invention, individual vascular networks that have been generated in step d) are extracted/isolated, e.g., transferred into one well of a low-attachment 96-well-plate, followed by culturing vascular networks in StemPro®-34 medium, supplemented with 25 ng/ml VEGF, 50 ng/ml SCF, 50 ng/ml IL-3, 50 ng/ml Flt-3L and 5 ng/ml TPO for about 6 to 8 days, preferably about 7 days. The medium is refreshed every about 3 to 4 days.

Then, the mature mammalian bone marrow organoids according to the present invention are produced. The present method advantageously uses Wnt-activators (e.g., CHIR99021) and Nodal-Inhibitors (e.g., SB431542) which are important for mesodermal patterning leading to induction of definitive hematopoiesis, and lineage-directed cytokines, such as EPO, IL-6 and G-CSF are avoided. Nevertheless, EPO may be used at a later stage in order to generate more mature erythroid cells.

Preferred is the method for producing mammalian bone marrow organoids according to the present invention, wherein the ratio of the 3D collagen I to Matrigel® in the matrix as used is between about 1:1 and 4:1, preferably about 3:1 or 1.2:1. The Matrigel®, in which the organoids are embedded, is derived from a mouse Engelbreth-Holm-Swarm sarcoma cell line, which hampers cGMP-compliant manufacturing of BMOs for the moment. Xenogeneic-free synthetic scaffold materials have been developed and tested for organoid differentiation (reviewed in Aisenbrey E A, Murphy W L. Synthetic alternatives to Matrigel. Nat Rev Mater. 2020 July; 5(7):539-51), which can be advantageously included in the method according to the present invention.

Further preferred is the method for producing mammalian bone marrow organoids according to the present invention, wherein the resuspension of the mesoderm-induced embryoid bodies comprises the use of a pipette.

Preferred is the method for producing mammalian bone marrow organoids according to the present invention, wherein the dissociating and/or extracting and isolating of individual vascular networks is mechanically, and preferably comprises the use of sterile dissection tools.

In the method for producing mammalian bone marrow organoids according to the present invention, the mammal is preferably selected from the group consisting of a human, a mouse, a monkey, a rat, a pig, a dog, a cat, a rabbit, a sheep, cattle, an equine, and a goat. Most preferred is a human, thus providing hiPSC.

Possible is also an implementation of the methods according to the present invention in a high-throughput format, for example in a 96-well-plate format.

The methods according to the present invention may furthermore comprise the step of testing and/or analyzing the BMOs as produced for their suitability as a pharmaceutical preparation for transplantation and other treatment purposes.

The methods according to the present invention may furthermore comprise the step of isolating cells and cell types from the BMOs as produced. In this context, the BMOs can be used as source of functional endothelial cells, pericytes, hematopoietic stem cells, and mesenchymal stem cells. Preferred is a method according to the present invention, further comprising the step of isolating mesenchymal stem cells (CD45⁻ CD31⁻ CD34⁻ CD90⁺ CD105⁺ CD271⁺ CD73⁺) or HSCs. This can be preferably done by cell sorting, e.g., FACS. In the context of the present invention, it was found that in trilineage differentiation assays. FACS-sorted MSCs had the capacity to differentiate into osteogenic, chondrogenic and adipogenic cells, which was visualized by Alizarin-Red-S, Alcian-blue and Oilred-O staining, respectively. Sorted BMO-derived MSCs expanded in culture and displayed serial replating capacity.

Yet another aspect of the present invention then relates to a preparation of HSCs or MSCs as produced.

Yet another aspect of the present invention then relates to a vascular network or mature mammalian bone marrow organoid, produced according to the method according to the present invention, or a pharmaceutical composition comprising the vascular network and/or mature mammalian bone marrow organoid.

Yet another aspect of the present invention then relates to an assembloid, comprising the vascular network or mature mammalian bone marrow organoid according to the present invention with at least one additional iPSC-derived organoid. Preferred is the assembloid according to the present invention, comprising immune cells, blood vessels, and pericytes.

Since the lack of immune cells and vasculature presents a common limitation of organoid models, the present invention includes the combination of the present BMOs with other iPSC-derived organoids to form so-called assembloids integrating immune cells, blood vessels and pericytes (see for example, Sharma, A., Sances, S., Workman, M. J. & Svendsen, C. N. Multi-lineage Human iPSC-Derived Platforms for Disease Modeling and Drug Discovery. Cell Stem Cell 26, 309-329 (2020)., Kanton, S. & Pa ca, S. P. Human assembloids. Development 149, dev201120 (2022)). While previous approaches required mixing these cell types (see, for example, Wang, L. et al. A human three-dimensional neural-perivascular ‘assembloid’ promotes astrocytic development and enables modeling of SARS-CoV-2 neuropathology. Nat. Med. 27, 1600-1606 (2021)), an iPSC-derived BMO system more appropriately models developmental and functional interactions in a spatial context. Also, the combination of bone marrow organoids with other organoids has the advantage that not only vascular cells but also hematopoietic cells are included.

Furthermore provided are the vascular network or mammalian bone marrow organoid, preferably a mature organoid, produced according to the method according to the present invention, an assembloid according to the present invention or a pharmaceutical composition comprising the vascular network and/or mature mammalian bone marrow organoid and/or the assembloid according to the present invention for use in the treatment of diseases.

Pharmaceutical compositions as used may optionally comprise a pharmaceutically acceptable carrier. The person skilled in the art knows suitable formulations for cells and cellular products and will readily be able to choose suitable pharmaceutically acceptable carriers or excipients, depending, e.g., on the formulation and administration route of the pharmaceutical composition.

Pharmaceutically acceptable carriers or excipients include diluents (fillers, bulking agents, e.g. lactose, microcrystalline cellulose), disintegrants (e.g. sodium starch glycolate, croscarmellose sodium), binders (e.g. PVP, HPMC), lubricants (e.g. magnesium stearate), glidants (e.g. colloidal SiO₂), solvents/co-solvents (e.g. aqueous vehicle, Propylene glycol, glycerol), buffering agents (e.g. citrate, gluconates, lactates), preservatives (e.g. Na benzoate, parabens (Me, Pr and Bu), BKC), anti-oxidants (e.g. BHT, BHA, Ascorbic acid), wetting agents (e.g. polysorbates, sorbitan esters), thickening agents (e.g. methylcellulose or hydroxyethylcellulose), sweetening agents (e.g. sorbitol, saccharin, aspartame, acesulfame), flavoring agents (e.g. peppermint, lemon oils, butterscotch, etc.), humectants (e.g. propylene, glycol, glycerol, sorbitol). Other suitable pharmaceutically acceptable excipients are inter alia described in Remington's Pharmaceutical Sciences, 15^{th} Ed., Mack Publishing Co., New Jersey (1991) and Bauer et al., Pharmazeutische Technologic, 5^{th} Ed., Govi-Verlag Frankfurt (1997).

The pharmaceutical composition can be administered in any suitable way, e.g. in the form of solutions, syrups, emulsions or suspensions. Administration is preferably carried out by transfusion, e.g., in the form of injections or infusions.

In addition to the aforementioned products of the invention, the pharmaceutical composition can contain further customary, usually inert carrier materials or excipients. Thus, the pharmaceutical preparations can also contain additives, such as, for example, fillers, extenders, disintegrants, binders, glidants, wetting agents, stabilizers, emulsifiers, preservatives, sweetening agents, colorants, flavorings or aromatizers, buffer substances, and furthermore solvents or solubilizers or agents for achieving a depot effect, as well as salts for changing the osmotic pressure, coating agents or antioxidants. They can also contain other therapeutically active substances as also described herein.

The present invention provides a vascularized BMO preparation or system (e.g. assembloid according to the present invention) derived exclusively from iPSCs (in particular hiPSCs), mimicking hematopoiesis in a multicellular context of native bone marrow, comprising blood cells and various bone marrow “niche cells”, such as endothelial cells and mesenchymal cells.

The preparations as produced can be generally used in two strategies; either as a research tool, for example to study the interaction between niche cells and hematopoietic cells in the pathogenesis of hematological diseases. The tool can also be used to study the effects of drugs on the system, and to use the effects as identified to develop and test new therapies. The tool can be further used to test cellular therapies, such as CAR-T-cells or antibodies or the combination of T-cells and antibodies (BiTe) against e.g. Leukemia Stem Cells (LSCs) or their effect on HSCs. Furthermore, the system can be used as a tool to screen and identify new drugs against hematological diseases and other diseases as disclosed herein. In the second strategy, the “products” and compositions as generated can be used themselves as therapeutics, i.e., to prevent and/or treat bone-marrow related diseases, such as hematological diseases and other diseases as disclosed herein. This strategy involves the possibility of a transplantation of human BMOs as produced or the improved in vitro generation of human blood cells from iPSCs, e.g., autologous patient-specific production, and subsequent infusion or transplantation. The present invention has the advantage to maintain the stemness of the hematopoietic stem cells, which is usually lost when cultured in vitro with cytokines. When stemness is preserved, one can perform single cell studies and select for stem cells that have the desired genetic modification.

Preferred is a vascular network or mature mammalian bone marrow organoid or an assembloid produced according to the present invention, which is a model for a bone marrow related disease, such as, for example a hematological disease, severe congenital neutropenia, myelofibrosis, blood cell cancers, anemia, thrombocytopenia, inborn errors of hematopoiesis and immunity, conditions related to HIV, sickle cell disease, and complications from chemotherapy or transfusions. In these cases, the model is generated on iPSCs that are mutated or modified in order to reflect the origin of the bone marrow related disease, and the cells are then used to generate the organoids. Nevertheless, also the organoids or assembloids or networks or certain cellular components thereof may be modified in order to reflect the origin of the bone marrow related disease to be analyzed.

Yet another aspect of the present invention thus relates to a method for identifying a pharmaceutically active compound against a bone marrow related disease, comprising the steps of a) providing a vascular network or mature mammalian bone marrow organoid or an assembloid produced according to the present invention, which is a model for at least one bone marrow related disease, b) contacting said vascular network or mature mammalian bone marrow organoid or the assembloid according to step a) with at least one potentially pharmaceutically active compound, and c) identifying a physiological effect reflecting or indicating a treatment or amelioration of said bone marrow related disease in the presence of said at least one potentially pharmaceutically active compound, when compared to the absence of said at least one potentially pharmaceutically active compound, or to a control, wherein said effect identifies a pharmaceutically active compound against a bone marrow related disease. The bone marrow related disease may be, for example, a hematological disease, severe congenital neutropenia, myelofibrosis, blood cell cancers, anemia, thrombocytopenia, inborn errors of hematopoiesis and immunity, conditions related to HIV, sickle cell disease, and complications from chemotherapy or transfusion.

Preferred is the method or use according to the present invention, wherein said contacting is in vivo or in vitro, in solution or comprises the candidate compound bound or conjugated to a solid carrier. Respective formats are also described in the art and known to the person of skill.

In the context of the present invention, the anti-senescence candidate compound can be selected from any suitable molecule that is suitable, such as a chemical organic molecule, a molecule selected from a library of small organic molecules (molecular weight less than 500 Da), a molecule selected from a combinatory library, a cell extract, in particular a plant cell extract, a small molecular drug, a protein, a protein fragment, a molecule selected from a peptide library, an antibody or fragment thereof.

These candidate molecules may also be used as a basis to screen for improved compounds, (see below).

In the context of the present invention, any method that is suitable for detecting the effect of the compound may be used. Respective methods are known to the person of skill and are disclosed in the art. The components of the assays as disclosed herein may be labelled, for example with a radiolabel or fluorescent label or with an antigenic label.

The present invention further relates to identifying improved compounds that have been identified in a first round of screening/identification. Following the provision of a compound as identified, the compound can be modified. In general, many methods of how to modify compounds of the present invention are known to the person of skill and are disclosed in the literature. Modifications of the compounds will usually fall into several categories, for example a) chemical modifications, e.g. through the addition of additional chemical groups, b) changes of the size, length and/or charge of the compound, and c) the attachment of additional groups to the molecule (including marker groups, labels, linkers or carriers, such as chelators). All these modifications present a new strategy wherein any one of these or a combination thereof finally leads to the “rational design” of improved molecules for use in the context of the present invention. The present invention also includes strategies in order to further improve compounds that have only partially undergone “directed evolution” or “directed mutagenesis”, i.e., the compounds can undergo several successive rounds of the above methods.

In a next step, the modified compound is tested for a change of the physiological effect reflecting or indicating a treatment or amelioration of said bone marrow related disease in the presence of said at least one potentially pharmaceutically active compound, when compared to the absence of said at least one potentially pharmaceutically active compound, compared to the non-modified pharmaceutically active compound or to a control.

Yet another aspect of the present invention thus relates to a pharmaceutically active compound against a bone marrow related disease as identified according to the present invention, or a pharmaceutical composition comprising the pharmaceutically active compound. This aspect also includes a method to produce a pharmaceutical composition comprising the pharmaceutically active compound against a bone marrow related disease as identified according to the present invention, comprising formulating the compound with a suitable diluent and/or carrier. In general, the same conditions apply to this pharmaceutical composition as mentioned above.

Yet another aspect of the present invention then relates to the use of the vascular network or mature mammalian bone marrow organoid or the assembloid or the pharmaceutical compositions (including the pharmaceutically active compound as identified) according to the present invention as a model system for identifying and/or testing pharmaceutically effective compounds for treating or preventing of a bone marrow related disease, such as, for example, a hematological disease, severe congenital neutropenia, myelofibrosis, blood cell cancers, anemia, thrombocytopenia, inborn errors of hematopoiesis and immunity, conditions related to HIV, sickle cell disease, and complications from chemotherapy or transfusions. The identifying and testing is generally outlined above.

Yet another aspect of the present invention then relates to the use of the vascular network or mature mammalian bone marrow organoid or the assembloid or the pharmaceutical compositions (including the pharmaceutically active compound as identified) according to the present invention as a model system in the pathogenesis of a bone marrow related disease, such as, for example, a hematological disease, severe congenital neutropenia, myelofibrosis, blood cell cancers, anemia, thrombocytopenia, inborn errors of hematopoiesis and immunity, conditions related to HIV, sickle cell disease, and complications from chemotherapy or transfusions. As mentioned above, in these cases, the model as used may be generated on iPSCs that are mutated or modified in order to reflect the origin of the bone marrow related disease, and the cells are then used to generate the organoids. Nevertheless, also the organoids or the assembloids or networks or certain cellular components thereof may be modified in order to reflect the origin of the bone marrow related disease to be analyzed. The model system may also be used to test the BMOs for their suitability as a pharmaceutical preparation for transplantation and other treatment purposes.

Yet another aspect of the present invention then relates to the use of the vascular network or mature mammalian bone marrow organoid or the assembloid or the pharmaceutical composition according to the present invention for the in vitro production of BMOs or mammalian blood cells, in particular autologous BMOs or mammalian blood cells, in particular for transplantation.

Yet another aspect of the present invention then relates to the use of the vascular network or mature mammalian bone marrow organoid or the assembloid according to the present invention to maintain stemness of hematopoietic stem cells (HSC). It is advantageous to keep HSC in vitro for further quality control studies and to sort only those cells for therapeutic application that show desired molecular changes without any unwanted side effects (e.g. off-target editing, or activation of oncogenes, or the like). The present invention has the advantage to maintain the sternness of the hematopoietic stem cells, which is usually lost when cultured in vitro with cytokines. When stemness is preserved, one can perform single cell studies and select for stem cells that have the desired genetic modification.

Yet another aspect of the present invention then relates to a pharmaceutically effective amount of the vascular network or mature mammalian bone marrow organoid or the assembloid or the pharmaceutical compositions (including the pharmaceutically active compound as identified) according to the present invention for use in the treatment or prevention of a bone marrow related disease, such as, for example, a hematological disease, severe congenital neutropenia, myelofibrosis, blood cell cancers, anemia, thrombocytopenia, inborn errors of hematopoiesis and immunity, conditions related to HIV, sickle cell disease, and complications from chemotherapy or transfusions, preferably for use in transplantation.

Yet another aspect of the present invention then relates to a method for preventing or treating a bone marrow related disease, such as, for example, a hematological disease, severe congenital neutropenia, myelofibrosis, blood cell cancers, anemia, thrombocytopenia, inborn errors of hematopoiesis and immunity, conditions related to HIV, sickle cell disease, and complications from chemotherapy or transfusions in a subject, comprising administering to said subject an effective amount of a vascular network or mature mammalian bone marrow organoid or the assembloid or the pharmaceutical compositions (including the pharmaceutically active compound as identified) according to the present invention. Preferably, said method comprises transplantation of an effective amount of a vascular network or mature mammalian bone marrow organoid or the assembloid according to the present invention.

It is to be understood that the products, compounds and/or a pharmaceutical composition are for use to be administered to a human patient. The term “administering” means administration of a sole therapeutic agent or in combination with another therapeutic agent. It is thus envisaged that the pharmaceutical compositions of the present invention are employed in co-therapy approaches, i.e. in co-administration with other medicaments or drugs and/or any other therapeutic agent which might be beneficial in the context of the methods of the present invention. Nevertheless, the other medicaments or drugs and/or any other therapeutic agent can be administered separately from the compound for use, if required, as long as they act in combination (i.e. directly and/or indirectly, preferably synergistically) with the present compound for use.

Thus, the compounds of the invention can be used alone or in combination with other active compounds—for example with medication and therapy already known for the treatment of the aforementioned diseases, whereby in the latter case a favorable additive, amplifying or preferably synergistically effect is noticed.

As mentioned herein, the product or compound is administered to said subject in an effective dosage. This dosage can vary within wide limits and is to be suited to the individual conditions in each individual case. For the above uses, the appropriate dosage will vary depending on the mode of administration, the particular condition to be treated and the effect desired. In general, however, satisfactory results are achieved at dosage rates are as above, e.g. of about 1 to 100 mg/kg animal body weight particularly 1 to 50 mg/kg. Suitable dosage rates for larger mammals, for example humans, are of the order of from about 10 mg to 3 g/day, conveniently administered once or in divided doses, e.g. 2 to 4 times a day, or in sustained release form. In general, a daily dose of approximately 10 mg to 100 mg, particularly 10 to 50 mg, per human individual is appropriate in the case of the oral administration. An effective concentration to be reached at the cellular level can be set at between 50 to 200 μM, preferably at about 100 μM.

Another aspect of the present invention then relates to a kit, for example a diagnostic kit comprising materials for performing a method according to the present invention. Materials are a set of media, such as the aggregation medium with Rho-associated protein kinase (ROCK)-inhibitor of step a) as herein, the serum-free, stabilized cell culture medium suitable for the feeder-free maintenance and expansion of human embryonic stem cells, supplemented in accordance with step b) as herein, Essential 6-medium supplemented in accordance with step c) as herein, and materials to produce the polymerized 3D collagen I/Matrigel® matrix as well as the StemPro®-34 medium supplemented in accordance with step d) as herein.

The kit may further comprise, relevant antibodies binding to cellular markers of the BMOs, dyes and other labels, as well buffers and matrices for performing the methods as above.

The kit may be used in the methods of the invention, i.e. for identifying and/or testing pharmaceutically effective compounds for treating or preventing of a bone marrow related disease, for use in a model system in the pathogenesis of a bone marrow related disease, and/or for the in vitro production of BMOs or mammalian blood cells, in particular autologous BMOs or mammalian blood cells, in particular for transplantation.

In summary, the present invention provides a vascularized BMO system derived exclusively from iPSCs and mimicking hematopoiesis in a multicellular context of native bone marrow.

Provided further is a method to generate complex self-organizing bone marrow organoids containing a de novo vascular network, hematopoietic cells and stromal niche cells by concomitant differentiation from human iPSCs. BMOs exhibit key cellular, structural and molecular features of the native human bone marrow niche. They contain not only multipotent hematopoietic stem and progenitors (HSPCs), but also mesenchymal stem and progenitor cells (MSPCs) and model developmental aspects of fetal bone marrow. This novel organoid system may serve studies of hematopoietic development and disease evolution. As mentioned above, provided further is a method to generate complex assembloids according to the present invention, e.g. (further) comprising iPSC-derived immune cells, blood vessels and pericytes.

The present invention relates to the following items.

Item 1. A method for producing mammalian bone marrow organoids, comprising a) generating embryoid bodies from substantially single induced pluripotent stem cells (iPSCs) obtained from at least one mammal, comprising culturing single iPSCs in an aggregation medium in the presence of at least one Rho-associated protein kinase (ROCK)-inhibitor for about 1 day; followed by culturing the embryoid bodies in a suitable serum-free, stabilized cell culture medium suitable for the feeder-free maintenance and expansion of human embryonic stem cells without ROCK-inhibitor for about 48 hours; b) inducing mesoderm in said embryoid bodies as formed in step a), comprising culturing said embryoid bodies in a suitable serum-free, stabilized cell culture medium suitable for the feeder-free maintenance and expansion of human embryonic stem cells, wherein the medium is supplemented with 80 ng/ml Bone morphogenetic protein 4 (BMP4), 4 μM of glycogen synthase kinase (GSK) 3 inhibitor, and 80 ng/ml Vascular Endothelial Growth Factor (VEGF), for about 48 hours, with resuspension of the mesoderm-induced embryoid bodies at about every 24 hours; c) replacing the medium of the culture of b) with Essential 6-medium, supplemented with 80 ng/ml VEGF, 25 ng/ml fibroblast growth factor (FGF)-2, 50 ng/ml stem cell factor (SCF), and 2 μM SB431542 for about 48 hours, with resuspension of the embryoid bodies at about every 24 hours; d) embedding of the mesoderm-induced embryoid bodies of step c) into a suitable polymerized 3D collagen I/Matrigel® matrix followed by overlaying the matrix with StemPro®-34 medium supplemented with 80 ng/ml VEGF, 25 ng/ml FGF-2, 50 ng/ml SCF and 2 μM SB431542 for about 48 hours, followed by cytokine replacement with 50 ng/mL VEGF, 50 ng/mL SCF, 50 ng/mL IL-3, 50 ng/mL Flt-3L, and 5 ng/mL TPO for about 48 hours, and a cytokine boost to 25 ng/mL VEGF, 50 ng/mL SCF, 50 ng/mL IL-3, 50 ng/mL Flt-3L, and 5 ng/mL TPO for about 48 hours; e) extracting individual vascular networks that have been generated in step d) followed by culturing in StemPro®-34 medium, supplemented with 25 ng/ml VEGF, 50 ng/ml SCF, 50 ng/ml IL-3, 50 ng/ml Flt-3L and 5 ng/ml TPO, with refreshing the medium every 3 to 4 days for about 7 to 11 days, whereby the mammalian bone marrow organoids are produced, and wherein preferably no lineage-directing cytokines, such as EPO, IL-6 and/or G-CSF, are used.

Item 2. The method for producing mammalian bone marrow organoids according to Item 1, further comprising the step of fixing the vascular networks in step e) and performing immunofluorescence analysis and/or dissociating the vascular networks into individual cells and performing flow cytometry analysis and/or live-cell imaging.

Item 3. The method for producing mammalian bone marrow organoids according to Item 1 or 2, further comprising the step of fixing the organoids as produced, and performing immunofluorescence analysis and/or dissociating the organoids into individual cells and performing flow cytometry analysis and/or live-cell imaging.

Item 4. The method for producing mammalian bone marrow organoids according to any one of Items 1 to 3, further comprising the step of culturing the organoids for a maximum of about 47 days, preferably about 60 days.

Item 5. The method for producing mammalian bone marrow organoids according to any one of Items 1 to 4, wherein the single iPSCs are provided by dissociating iPSC cells into single cells with Accutase® digestion.

Item 6. The method for producing mammalian bone marrow organoids according to any one of Items 1 to 5, wherein the ROCK-inhibitor is selected from the group consisting of Y-27632 ((1R,4r)-4-((R)-1-aminoethyl)-N-(pyridin-4-yl)cyclohexanecarboxamide), and fasudil.

Item 7. The method for producing mammalian bone marrow organoids according to any one of Items 1 to 6, wherein the aggregation medium is KnockOut DMEM/F12 with 20% serum substitute, 1% L-glutamine, 1% non-essential amino acids, 1% penicillin-streptomycin, and 100 μM β-mercaptoethanol.

Item 8. The method for producing mammalian bone marrow organoids according to any one of Items 1 to 7, wherein the serum-free, stabilized cell culture medium suitable for the feeder-free maintenance and expansion of human embryonic stem cells is mTeSR® Plus medium.

Item 9. The method for producing mammalian bone marrow organoids according to any one of Items 1 to 8, wherein the glycogen synthase kinase (GSK) 3 inhibitor is CHIR99021.

Item 10. The method for producing mammalian bone marrow organoids according to any one of Items 1 to 9, wherein the ratio of the 3D collagen I/Matrigel® matrix is between about 1:1 and 4:1, preferably about 3:1 or 1.2:1.

Item 11. The method for producing mammalian bone marrow organoids according to any one of Items 1 to 10, wherein the resuspension of the embryoid bodies/mesoderm-induced embryoid bodies comprises the use of a pipette.

Item 12. The method for producing mammalian bone marrow organoids according to any one of Items 1 to 11, wherein the dissociating or extracting of individual vascular networks comprises extraction with the use of sterile dissection tools.

Item 13. The method for producing mammalian bone marrow organoids according to any one of Items 1 to 12, wherein the mammal is selected from the group consisting of a human, a mouse, a monkey, a rat, a pig, a dog, a cat, a rabbit, a sheep, cattle, an equine, and a goat.

Item 14. A method for producing mesenchymal stem cells (MSCs) and/or hematopoietic stem cells (HSCs), comprising performing a method according to any one of Items 1 to 13, and suitably isolating the MSCs or HSCs from the vascular network or mammalian bone marrow organoid.

Item 15. A vascular network or mature mammalian bone marrow organoid, produced according to the method according to any one of Items 1 to 13, or a pharmaceutical composition comprising the vascular network and/or mature mammalian bone marrow organoid.

Item 16. An assembloid, comprising the vascular network or mature mammalian bone marrow organoid according to Item 15 with at least one additional iPSC-derived organoid.

Item 17. The assembloid according to Item 16, comprising immune cells, blood vessels, and pericytes.

Item 18. Use of the vascular network or mature mammalian bone marrow organoid according to Item 15 or the assembloid according to Item 16 or 17 as a model system for a bone marrow related disease, such as, for example a hematological disease, severe congenital neutropenia, myelofibrosis, blood cell cancers, anemia, thrombocytopenia, inborn errors of hematopoiesis and immunity, conditions related to HIV, sickle cell disease, and complications from chemotherapy or transfusions.

Item 19. Use of the vascular network or mature mammalian bone marrow organoid or the pharmaceutical composition according to Item 15 or the assembloid according to Item 16 or 17 as a model system in the pathogenesis of a bone marrow related disease, such as, for example, a hematological disease, severe congenital neutropenia, myelofibrosis, blood cell cancers, anemia, thrombocytopenia, inborn errors of hematopoiesis and immunity, conditions related to HIV, sickle cell disease, and complications from chemotherapy or transfusions.

Item 20. Use of the vascular network or mature mammalian bone marrow organoid or the pharmaceutical composition according to Item 15 or the assembloid according to Item 16 or 17 as a model system for identifying and/or testing pharmaceutically effective compounds for treating or preventing of a bone marrow related disease, such as, for example, a hematological disease, severe congenital neutropenia, myelofibrosis, blood cell cancers, anemia, thrombocytopenia, inborn errors of hematopoiesis and immunity, conditions related to HIV, sickle cell disease, and complications from chemotherapy or transfusions.

Item 21. Use of the vascular network or mature mammalian bone marrow organoid or the pharmaceutical composition according to Item 15 for the in-vitro production of BMOs or mammalian blood cells, in particular autologous BMOs or mammalian blood cells, in particular for transplantation.

Item 22. A pharmaceutically effective amount of the vascular network or mature mammalian bone marrow organoid or the pharmaceutical composition according to Item 15 or the assembloid according to Item 16 or 17 for use in the treatment of a bone marrow related disease, such as, for example, a hematological disease, severe congenital neutropenia, myelofibrosis, blood cell cancers, anemia, thrombocytopenia, inborn errors of hematopoiesis and immunity, conditions related to HIV, sickle cell disease, and complications from chemotherapy or transfusions, preferably for transplantation.

Item 23. MSCs or HSCs as produced according to item 14 or a pharmaceutical composition comprising the MSCs or HSCs.

Item 24. A pharmaceutically effective amount of the MSCs or HSCs or the pharmaceutical composition according to Item 23 for use in the treatment of a bone marrow related disease, such as, for example, a hematological disease, severe congenital neutropenia, myelofibrosis, blood cell cancers, anemia, thrombocytopenia, inborn errors of hematopoiesis and immunity, conditions related to HIV, sickle cell disease, and complications from chemotherapy or transfusions, preferably for transplantation.

The invention will now be further described in the following examples and with reference to the accompanying figures and the sequence listing, without being limited thereto. For the purposes of the present invention, all references as cited herein are incorporated by reference in their entireties.

FIG. 1 shows the generation of human iPSC-derived bone marrow organoids and analysis of cellular composition by flow cytometry. a) Schematic illustration of the workflow for BMO generation. b) Representative bright field images of fibroblast-derived iPSC (FiPSC) embryoid bodies on day 0 and day 4, sprouting embryoid bodies on day 6 and differentiated BMOs on day 17. c) Joint tSNE visualization of flow cytometric data of three independent differentiations resulting in 105,000 cells colored by protein expression. d) Overlay of manually gated populations on tSNE projection. Subsets defined as CD45⁻CD31⁺ endothelial, CD45⁺ hematopoietic, CD45⁺CD11b⁺ myeloid and CD45⁻CD31⁻CD271⁺ mesenchymal cells. CD45⁺CD11b⁻CD34⁺ as HSPCs and CD45⁻CD31⁻CD271⁺CD90⁺CD105⁺CD73⁺ as MSPCs. e) Frequencies of manually gated cell types, n=5 independent differentiations. f) Bright field microscopy of sorted and May-Gruenwald-Giemsa stained HSPCs and CD45⁺CD11b⁺ myeloid cells. Typical high nuclear-to-cytoplasm ratio of HSPCs. Morphology of sorted MSPCs in culture by phase-contrast. Representative images of n=6 experiments with two iPSC cell lines. iPSC, induced pluripotent stem cell; EB, embryoid body. Scale bars in b) 500 μm, day 6 inset 100 nm, f) 20 μm.

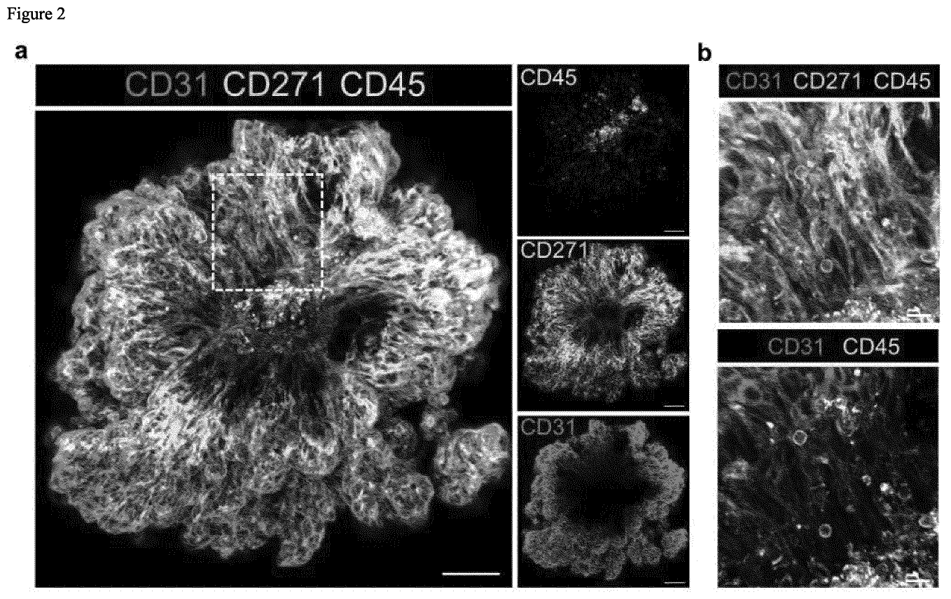

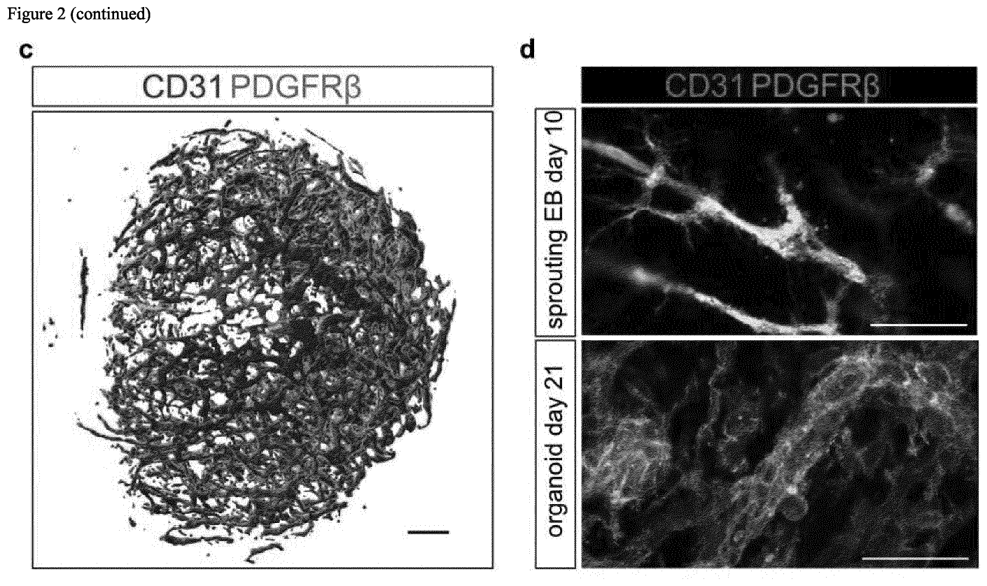

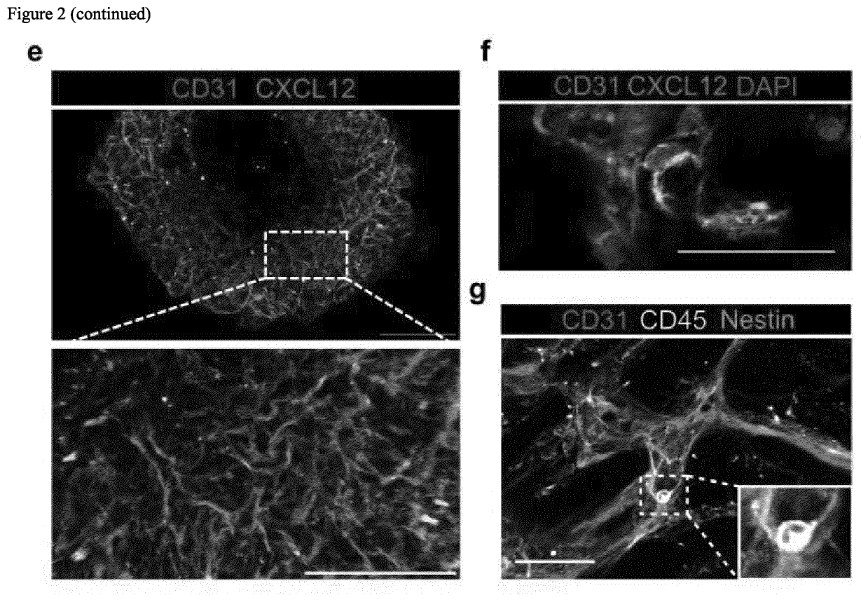

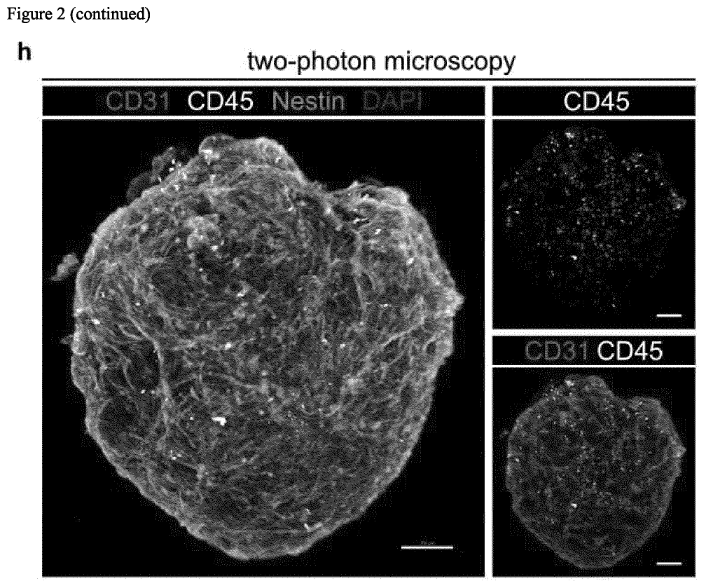

FIG. 2 shows that the spatial architecture of BMOs recapitulates key features of the human bone marrow niche. a) Confocal imaging of whole mount organoid reveals dense interconnected network of CD31-expressing vascular and CD271-expressing stromal cells containing hematopoietic cells marked by CD45. b) Magnified view of a) displaying CD45⁺ cells within CD31⁺ and CD271⁺ niche cells. c) Surface rendering of 3D z-reconstruction of organoid stained for endothelial cells (CD31) and pericytes (PDGFRβ) imaged by confocal microscopy to visualize vascular network and PDGFRβ⁺ pericytes. d) Pericyte-endothelial cell association on day 10 and day 21 of differentiation. Top: Finger-like extensions of PDGFRβ⁺ pericytes on day 10 of differentiation. Bottom: Tight association of a PDGFRβ⁺ pericyte with an endothelial cell on day 21 of differentiation. e) Niche marker CXCL12 is expressed in specific cell subsets throughout the organoid visualized by confocal microscopy. f) CXCL12-expressing pericytes in close association to endothelial cells. g) Nestin⁺ cells lining CD31⁺ vessels and CD45⁺ hematopoietic cells. Note CD45^{high} expressing cell with banded nucleus. h) Two-photon microscopy shows spatial architecture of whole organoid. Expression of key niche marker Nestin in spatial association with CD31⁺ vessels and CD45⁺ hematopoietic cells. Z-dimension 515 μm. Scale bars in a) 100 μm, b) 20 μm, c) 100 μm, d), e), f), g) 50 μm, h) 200 μm.

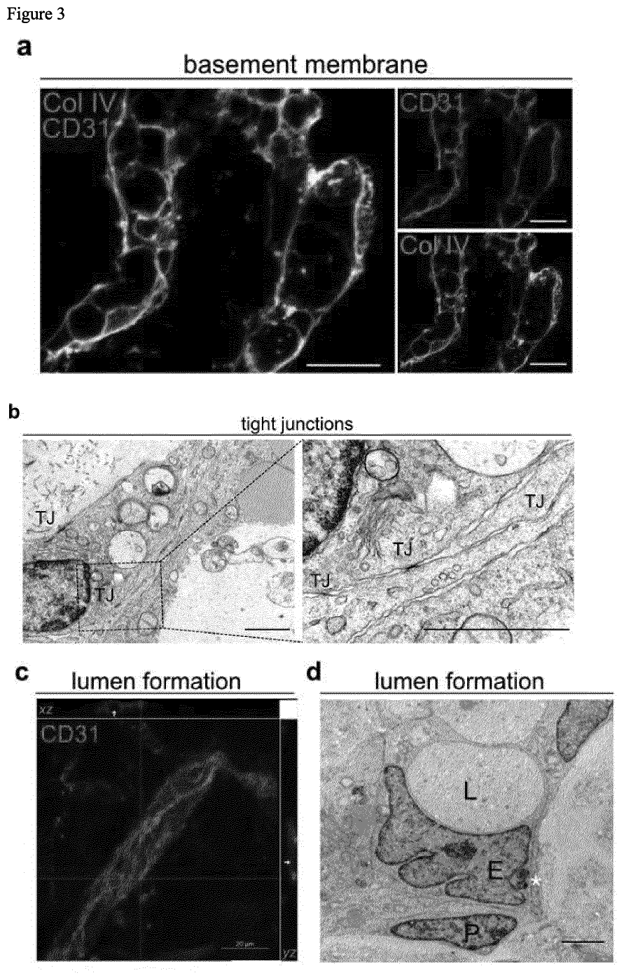

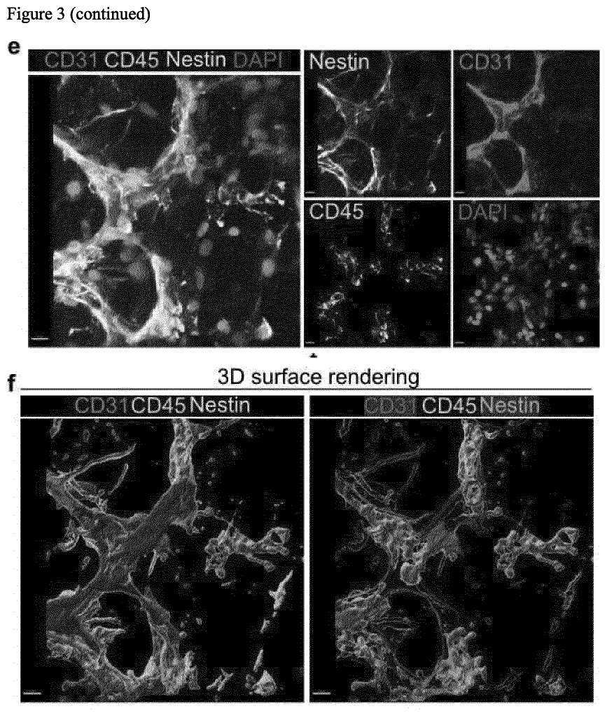

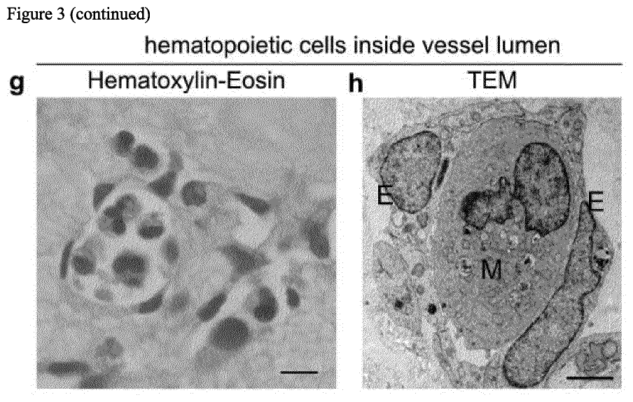

FIG. 3 shows vascular structures exhibiting key features of blood vessels and enclosing hematopoietic cells. a) Endothelial cells are covered by a Collagen IV⁺ (Col IV) basement membrane. b) TEM image of pericyte and endothelial cell connected by tight junctions (TJ). c) Orthogonal 2D z-projection of vessel structure of bottom image in FIG. 2d) in xz and yz direction. Note lumen formation indicated by arrows. d) TEM image of organoid section showing capillary-like structure by an endothelial cell (E) forming a lumen (L) and enclosed by a pericyte (P). Asterisk marking Weibel-Palade bodies. e) Confocal imaging of CD31⁺ vessels lined by Nestin expressing cells and enclosing CD45⁺ hematopoietic cells. f) Surface rendering of fluorescent image in e) to display vessel structures. Transparent surfaces for CD31 and Nestin reveal CD45⁺ cells inside the vessels. g) Histological sections reveal morphologically hematopoietic cells within lumen of vessel-like structures by Hematoxylin-Eosin (HE) stain. h) TEM image of organoid section showing endothelial cells (E) encompassing round cells resembling myeloid cells (M). Scale bars in a) 10 μm, b) 1 μm, c), 20 μm d) 2 μm, e), f), 10 μm, g) 20 μm, h) 2 μm.

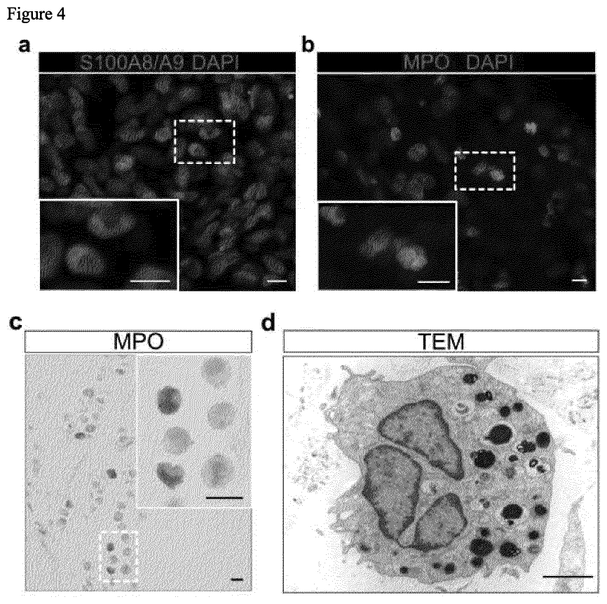

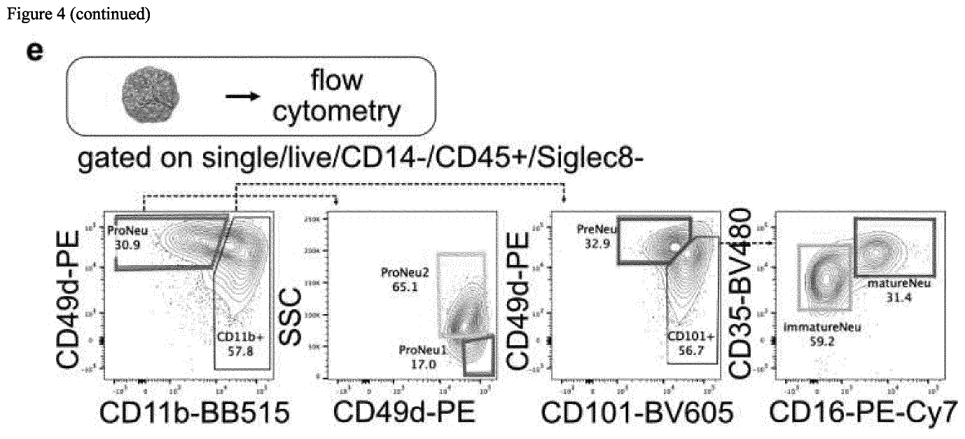

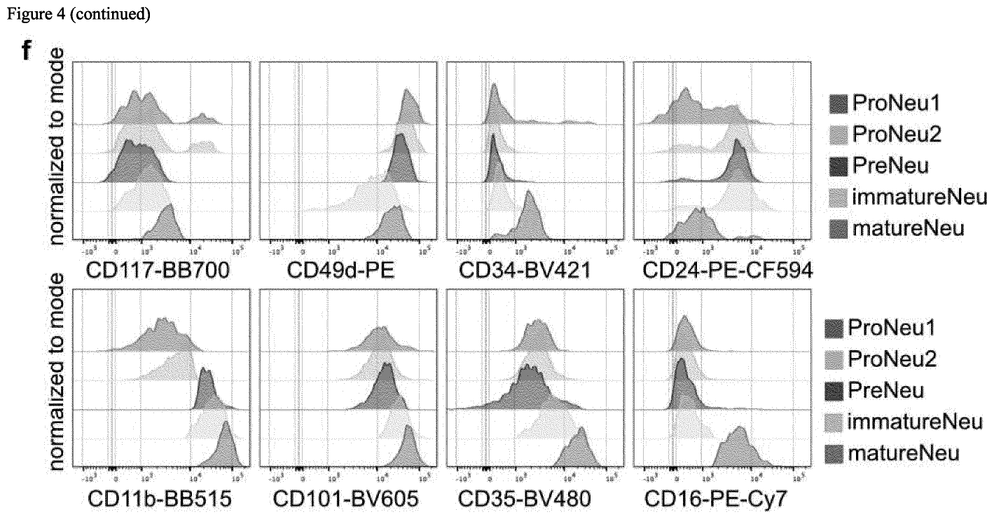

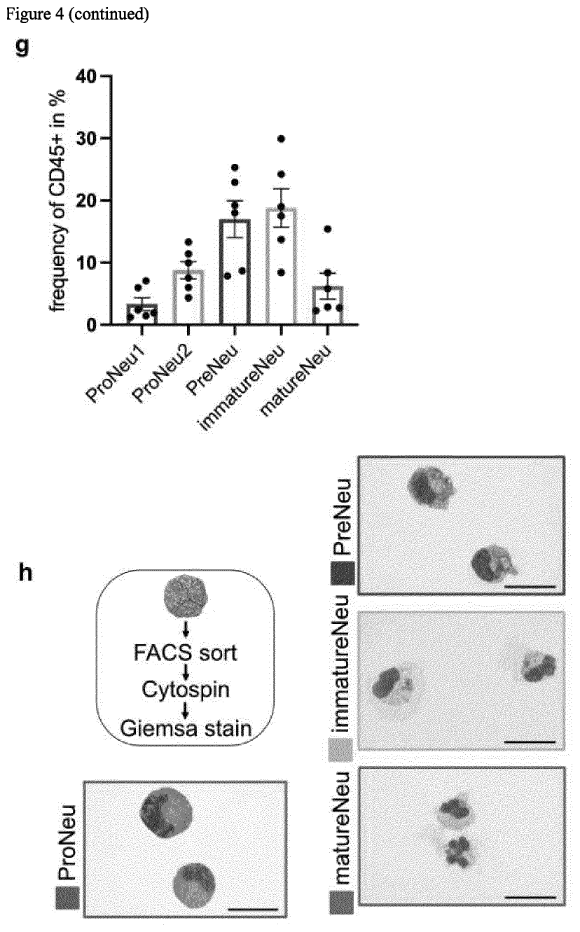

FIG. 4 shows that bone marrow organoids enable granulopoiesis. a) Expression of S100A8/A9 and b) Myeloperoxidase (MPO) in cells with banded/segmented nucleus. Magnified images of the rectangle areas are depicted in the insets. c) MPO expression confirmed by immunohistochemistry of organoid section. Magnified image of the rectangle area depicted in inset. d) Representative TEM image of typical morphology of a cell resembling a neutrophil granulocyte within the organoid. e) Gating scheme for neutrophil differentiation analysis of dissociated BMOs on day 21 of differentiation by flow cytometry. f) Histogram of surface marker expression of distinct neutrophil progenitor stages until mature neutrophil-like state of BMO-derived neutrophils. g) Frequency of neutrophil progenitor subpopulations within CD45⁺ population, n=6 independent experiments. h) May-Gruenwald-Giemsa staining of BMO-derived sorted neutrophil progenitors shows high morphological resemblance to human neutrophil progenitors in vivo. Scale bars a), b), 10 μm, c) 50 μm, inset 10 μm, d) 2 μm, h) 20 μm.

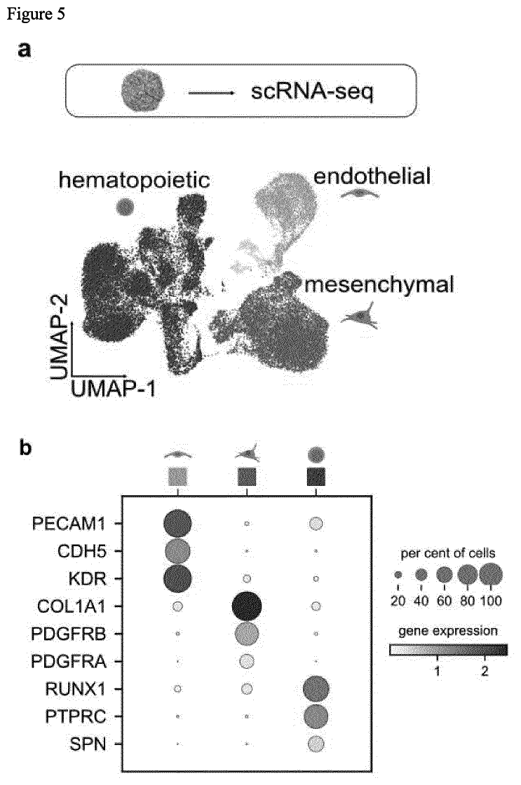

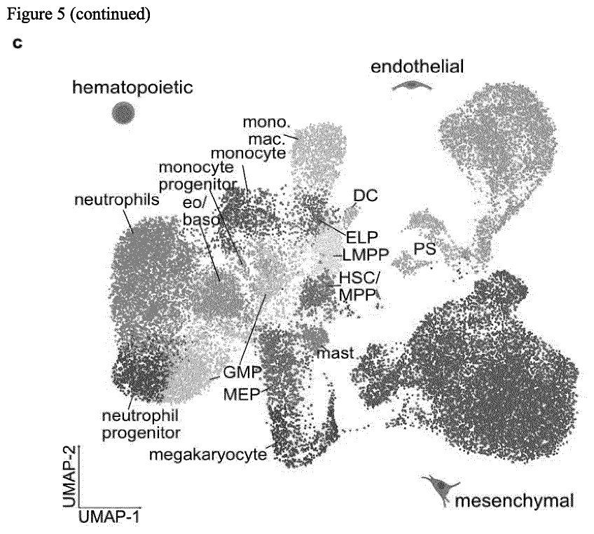

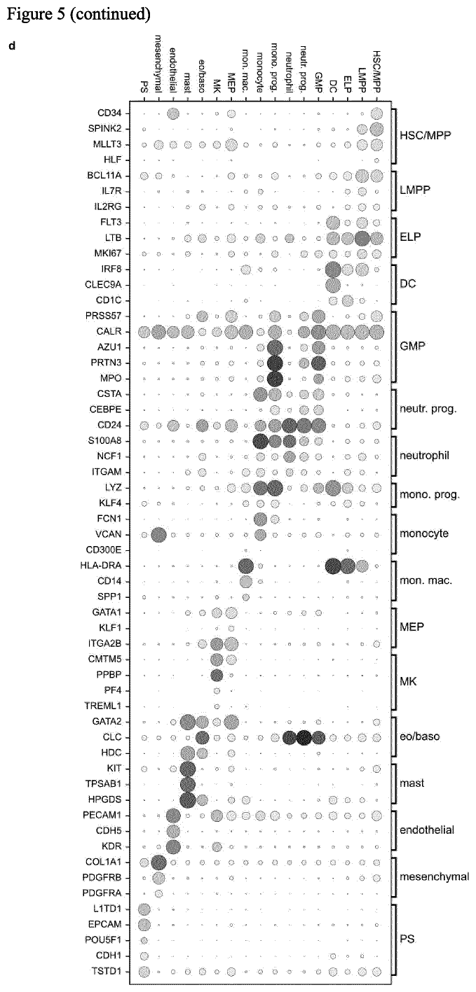

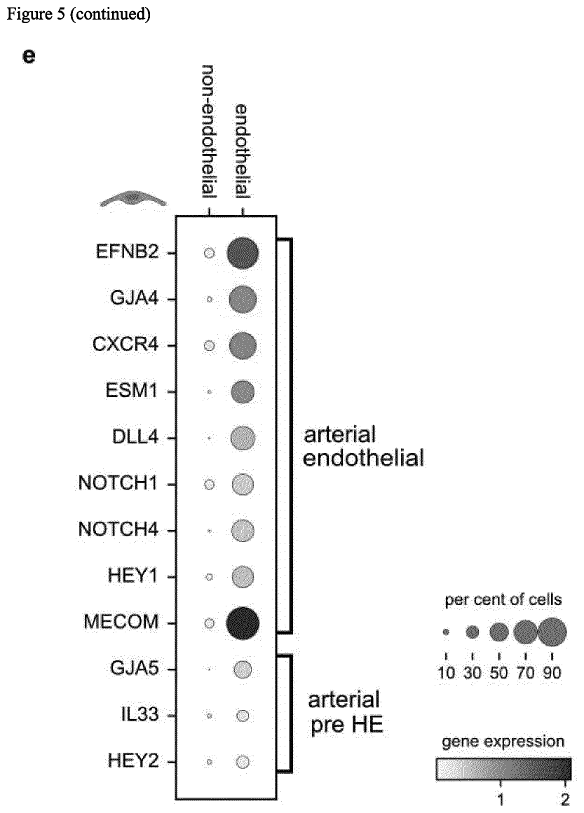

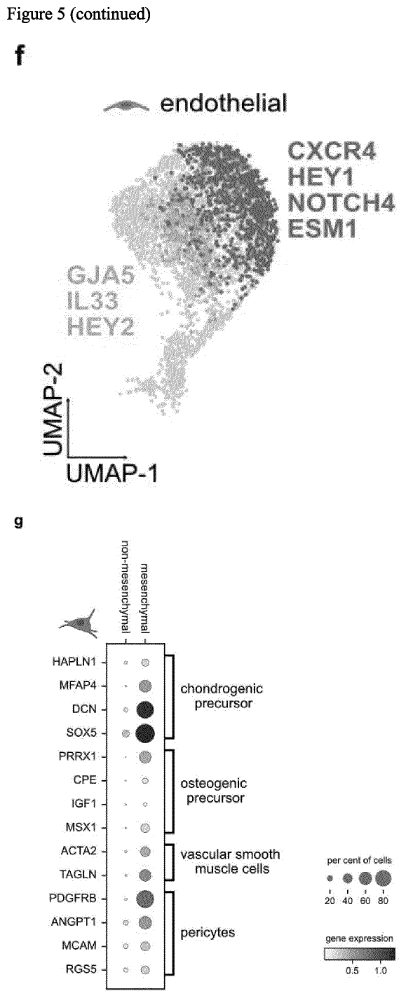

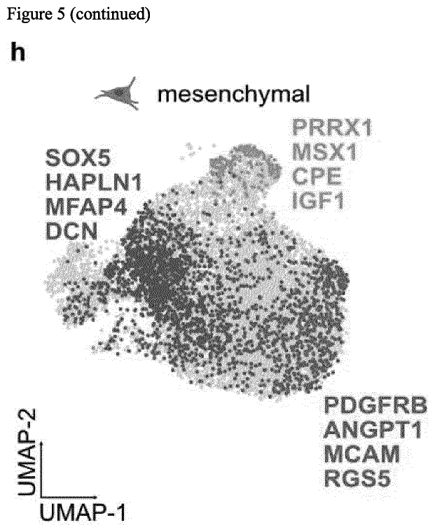

FIG. 5 shows the single-cell transcriptomic analysis of BMOs identifies diverse cell populations. a) Coarse grained clustering of scRNA-seq data reveals three main populations comprising endothelial, hematopoietic and mesenchymal cells. b) Expression of characteristic markers that are indicative of the three main populations. c) UMAP projection of a total number of 31,040 cells colored according to detailed cell type annotations. d) Expression of marker genes for annotation of hematopoietic lineages. e) Expression of marker genes for endothelial subtypes. f) UMAP projection for arterial endothelial and pre HE cells co-expressing displayed marker genes. g) Expression of marker genes for mesenchymal cell clusters. h) UMAP projection of distinct mesenchymal subsets co-expressing marker genes. DC, dendritic cell; ELP, early lymphoid progenitor; eo/baso, eosinophil/basophil; GMP, granulocyte/monocyte-progenitor; HE, hemogenic endothelium; HSC/MPP, hematopoietic stem cell/multipotent progenitor; LMPP, lymphoid-primed multipotent progenitors; MEP, megakaryocyte/erythroid progenitor, MK, megakaryocyte; mono. mac., monocytoid macrophage; PS, primitive streak-like.

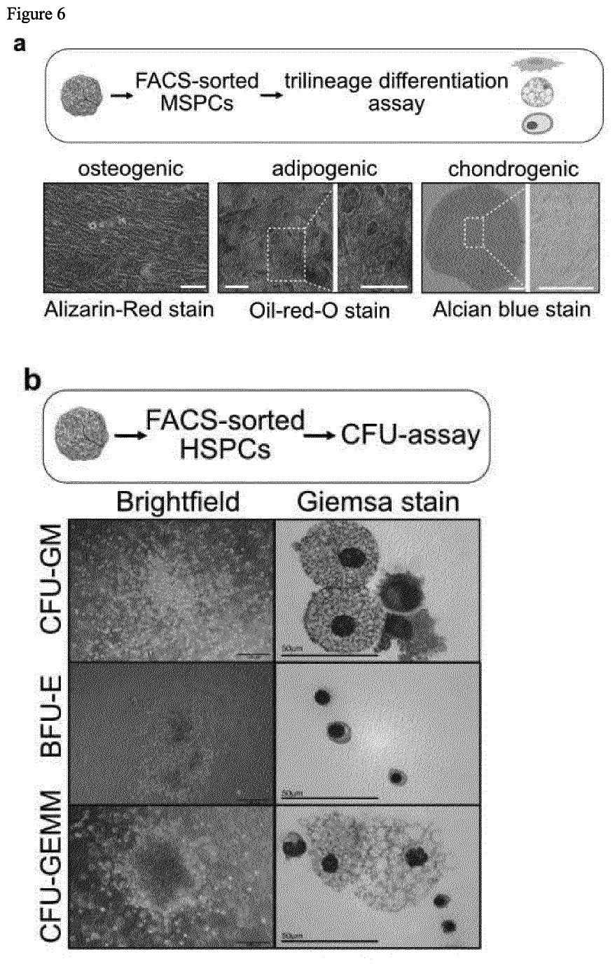

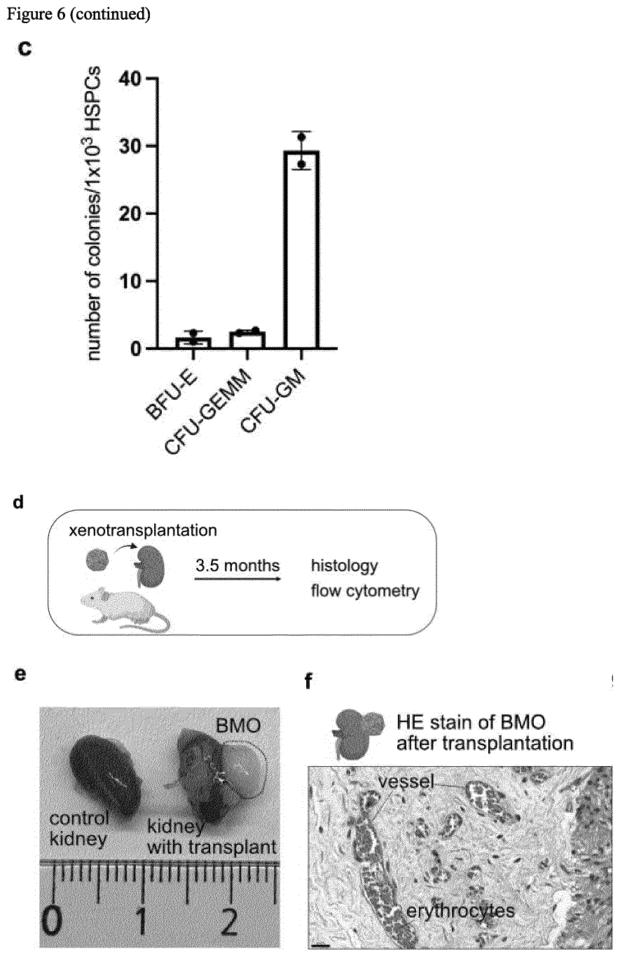

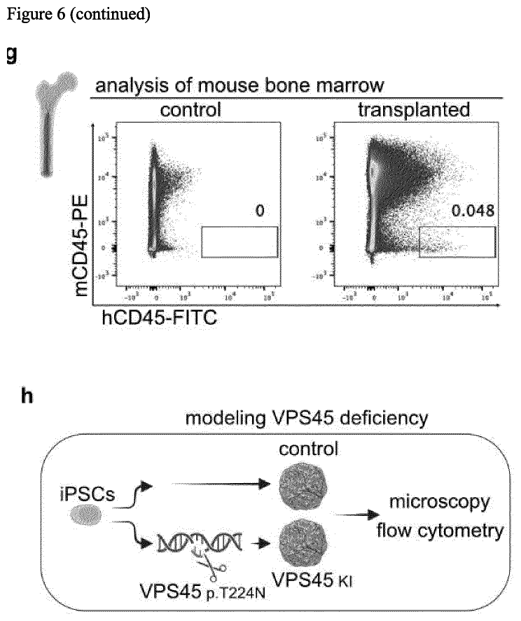

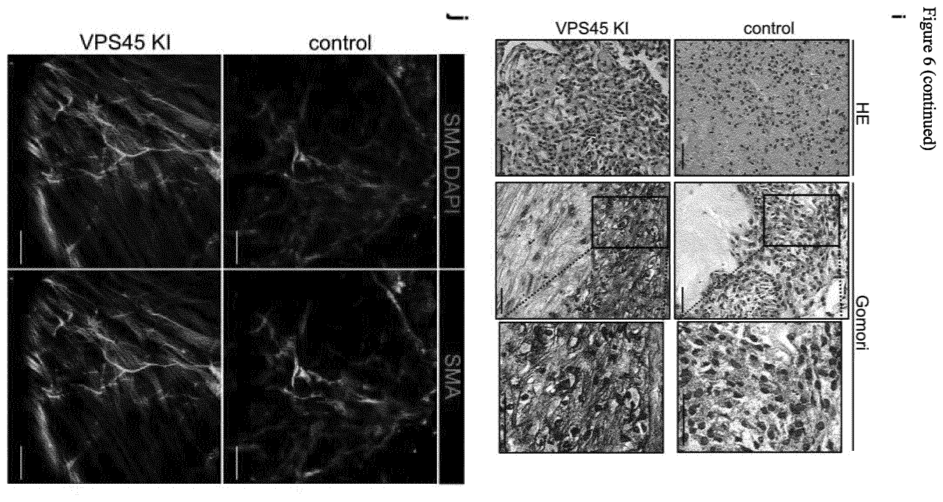

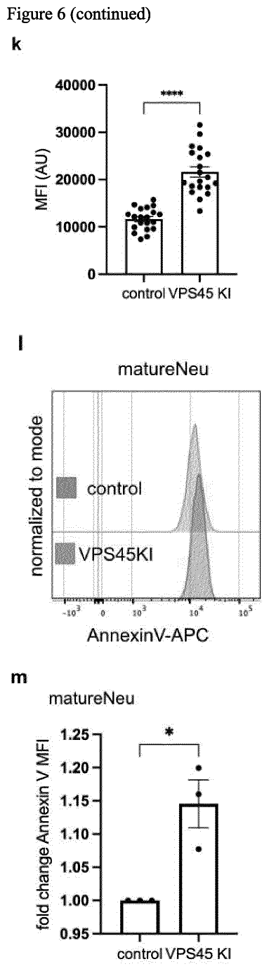

FIG. 6 shows functional properties of BMOs in vitro and in vivo and modeling of monogenic bone-marrow failure syndrome. a) Trilineage differentiation assay of FACS-sorted BMO-derived MSPCs. MSPCs were cultured in either osteogenic, adipogenic or chondrogenic differentiation medium for at least 21 days. Osteogenic, adipogenic and chondrogenic differentiation was visualized by Alizarin-Red-S, Oilred-O or Alcian-Blue staining, respectively; n=4 independent experiments with two iPS cell lines. b) Colony-forming unit assay of FACS-sorted BMO-derived CD45⁺CD11b⁻CD34⁺ HSPCs. CFU-GM, granulocyte-macrophage colony-forming unit, BFU-E, erythroid burst-forming unit, CFU-GEMM, granulocyte, erythrocyte, monocyte, megakaryocyte colony-forming unit; Representative phase contrast microscopy of colony morphology (left) and May-Gruenwald-Giemsa stain of picked colonies (right). c) Frequencies of BMO HPSC-derived colonies, n=4 independent experiments with two iPS cell lines, numbers for FiPSC-derived are displayed. d) Xenotransplantation of BMOs under the kidney capsule of NSG mice. e) Macroscopic morphology after 3.5 months showing further growth in vivo. f) Hematoxylin-Eosin-stained section of paraffin-embedded organoid 3.5 months after transplantation showing blood vessels filled with erythrocytes within the BMO. g) Representative plot for flow cytometric analysis reveals human CD45⁺ inside murine bone marrow of transplanted mice, but not in control mice, n=2 mice. h) Modeling of VPS45 deficiency in bone marrow organoids. Schematic overview of gene-editing and experimental set-up to create a VPS45-mutant iPS cell line with isogenic background. i) Histological comparison of control and VPS45 mutant BMOs by HE and Gomori stain reveals reticulin fibrosis in VPS45 mutant BMOs. n=8 organoids for each condition of two batches. j) Alpha smooth-muscle actin (SMA) expression in control and VPS45-mutant BMOs analyzed by immunofluorescence. k) Quantification of mean fluorescence intensity (MFI) of SMA expression, four different regions of n=5 organoids per condition of two batches. 1) Flow cytometry shows increased expression of Annexin V on matureNeus in VPS45 mutant BMOs, n=3. m) Quantification of Annexin MFI on matureNeus, n=3, * p<0.05, **** p<0.0001, unpaired two-tailed t-test. Scale bars in a) 100 μm b) 200 μm (left) and 50 μm (right), f) 20 μm, i), j) 50 μm.

### EXAMPLES

### Background

A 2D-system for the hematopoietic differentiation was described in 2011, wherein the blood precursor cells were generated from human induced pluripotent stem cells (hiPSCs) through the sequential induction of cytokines (8). At that time, a serum-free monolayer culture that can trace the in vivo hematopoietic pathway from ES/iPS cells to functional definitive blood cells via mesodermal progenitors was established, and stepwise tuning of exogenous cytokine cocktails induced the hematopoietic mesodermal progenitors via primitive streak cells. These progenitors were then differentiated into various cell lineages depending on the hematopoietic cytokines present.

The protocol of (8) was modified considerably in order to differentiate the hematopoietic progenitors into functional neutrophils and macrophages. For the generation of a vascularized bone marrow organoid, the inventors implemented parts of a protocol for the generation of vascular organoids recently published by Wimmer and colleagues (9). These hiPSC-derived vascular organoids were not only composed of functional endothelial cells and pericytes, but also contained very small numbers of hematopoietic cells and mesenchymal stromal cells (10).

The inventors concluded that the association of the above hematopoietic differentiation protocol with a three-dimensional vascularized culture system would promote the formation of complex cell interactions, thus leading to the formation of a structurally organized microenvironment resembling the natural bone marrow niche., which surprisingly turned out to be correct.

### Summary of the Preferred Method According to the Present Invention

Bone marrow organoids were generated from iPSCs by mesoderm induction and subsequent induction of hematopoiesis in a 3D matrix cytokine induction system. Human iPSC embryoid bodies were cultured in low-adhesion plates with Y-27632 and grown for 2-3 days until aggregates reached a size of 0.5 μm (FIG. 1A). Mesoderm was induced on day 0 with bone morphogenic protein 4 (BMP4), the GSK-3 inhibitor CHIR99021 (Sigma), and vascular endothelial growth factor (VEGF) and patterned on day 2 and 4 with VEGF, TGF-β RI Kinase Inhibitor VI SB431521 (Sigma), fibroblast growth factor 2 (FGF2) and stem cell factor (SCF). On day 4, the embryoid bodies were embedded in a collagen I/Matrigel® (Sigma) solution to induce vascular sprouting and promote cell organization in 3-dimensional form. Thereafter, hematopoietic differentiation was induced with VEGF, SCF, Fms Related Receptor Tyrosine Kinase 3 Ligand (Flt-3L), Interleukin 3 (IL-3) and Thrombopoietin (TPO). On day 10, the vascular networks were separated/extracted, and the individual networks transferred to a 96-well low attachment plate. From day 10 to day 17, the vascular networks formed into spherical BMOs and were collected on day 17, day 21 and day 45 for further analysis. The resulting BMOs were characterized by histological methods, confocal microscopy, flow cytometry and differentiation assays (FIG. 1).

### Detailed Preferred Method for Producing the Bone Marrow Organoids

Bone marrow organoids (BMO) were generated in a 3D-culture system using the sequential addition of growth factors as follows.

As exemplary shown on FIG. 1, on day −3 of the method, embryoid bodies were generated by dissociating iPSCs into single cells with Accutase® (Sigma) for 5 minutes at 37° C. Cell aggregates were mechanically disrupted using a P1000 pipette. The dissociation reaction was stopped with mTeSR® Plus (Fisher Scientific), and then the cells were collected at 300 g for 3 minutes at room temperature (RT).

The cells were resuspended in aggregation medium (KnockOut DMEM/F12 with 20% serum substitute, 1% L-glutamine, 1% non-essential amino acids, 1% penicillin-streptomycin and 100 μM β-mercaptoethanol) and the cells were treated with a counted by hemocytometer. 2.5-3×10⁶ cells were resuspended in aggregation medium supplemented with 50 μM Y-27632 and seeded in a low adherence petri dish.

Mesoderm was induced on day 0 with mTeSR® Plus supplemented with 80 ng/ml BMP4, 4 μM CHIR99021 and 80 ng/ml VEGF. For the medium change, the embryoid bodies were collected in 15 ml canonical tubes by gravity (15-30 minutes). To avoid excessive fusion, the embryoid bodies were resuspended once a day and placed on a rocking shaker. On day 2, the medium was changed to Essential 6 medium supplemented with 80 ng/mL VEGF, 25 ng/mL FGF-2, 50 ng/mL SCF, and 2 μM SB431542.

On day 4, 30-60 embryoid bodies were embedded in a 12-well plate with 1 ml/well of a collagen I/Matrigel® mixture. Collagen I solution was prepared according to manufacturer's protocol (50 μl 10×DMEM, 137.5 μl ddH2O, 12.5 μl 7.5% sodium bicarbonate, 235.9 μl Hams-F12, 9.45 μl HEPES, 4.6 μl Glutamax, 300 μl 5 mg/ml collagen type I) and 1N NaOH was added dropwise to bring the solution to pH 7.4.

For embedding, a layer of 500 l/well of collagen I-Matrigel® mixture was first prepared and polymerized at 37° C. for 1 hour to prevent the embryoid bodies from sinking to the bottom of the dish. The embryoid bodies were then resuspended in 500 l/well of the collagen-I-Matrigel mixture and a second layer was prepared, which was polymerized at 37° C. for 1 hour. Finally, the embedded embryoid bodies were overlaid with prewarmed StemPro®-34 medium supplemented with 80 ng/ml VEGF, 25 ng/ml FGF-2, 50 ng/ml SCF and 2 μM SB431542.

On day 6, cytokines were replaced with 50 ng/mL VEGF, 50 ng/mL SCF, 50 ng/mL IL-3, 50 ng/mL Flt-3L, and 5 ng/mL TPO. On day 8, cytokines were changed to 25 ng/mL VEGF, 50 ng/mL SCF, 50 ng/mL IL-3, 50 ng/mL Flt-3L, and 5 ng/mL TPO.

On day 10, the vascular networks that had arisen from individual embryoid bodies were extracted with sterile dissection tools under the laminar flow unit with the lowest magnification of an inverted light microscope. Individual vascular networks were then transferred and further cultured in a 96-well low attachment plate. Thereafter, the organoids were cultured in StemPro®-34 medium (Fisher Scientific) supplemented with 25 ng/ml VEGF, 50 ng/ml SCF, 50 ng/ml IL-3, 50 ng/ml Flt-3L and 5 ng/ml TPO, with half of the medium every 3 4 days refreshed. At day 10, the vascular networks and at day >17 the mature organoids were either fixed for immunofluorescence analysis or dissociated into individual cells for flow cytometry analysis. In this case, the organoids were cultured until day 45.

### Heterogenous Cell Type Composition

In order to dissect the cell type composition, the inventors analyzed dissociated BMOs at day 17 of differentiation by flow cytometry (FIG. 1c). To visualize subset heterogeneity the inventors performed dimensionality reduction of the flow cytometric data by t-Distributed Stochastic Neighbor Embedding (t-SNE) (FIG. 1a). The inventors integrated data of three independent differentiations into a joint t-SNE visualization to create a representative map across different experiments (FIG. 1a). Overlay of manually gated populations revealed three main cell clusters (FIG. 1e). Based on CD45 as pan-leukocyte marker, CD31 (PECAM1) as a marker for vascular endothelial cells (ECs) and CD271 (NGFR) as marker for mesenchymal cells, these clusters were assigned as endothelial (CD45−CD31+), hematopoietic (CD45+) and mesenchymal stromal cell clusters (CD45−CD31−CD271+), respectively.

Hematopoietic cells were further defined as HSPCs (CD45+CD11b−CD34+) and myeloid cells (CD45+CD11b+). CD45−CD31−CD271+CD90+CD105+CD73+ cells were characterized as MSPCs according to the minimal criteria for multipotent MSPCs. Quantitative analysis of the cellular composition on day 17 of culture yielded an average content of 39.3% hematopoietic cells, 41.3% mesenchymal cells, 6% ECs, 1.42% HSPCs and 0.96% MSPCs per BMO (n=5). This relative cell type composition was constant at different time points of differentiation. To determine the reproducibility of the protocol, the inventors also generated BMOs by using an iPS cell line derived from exfoliated renal epithelial cells present in urine, so-called urinary iPSCs (UiPSCs). The UiPSC-derived BMOS showed a highly similar morphology to fibroblast-derived iPSC (FiPSCs). The main composition of hematopoietic, mesenchymal and endothelial cells was consistent, yet UiPSC-derived BMOs showed a higher percentage of endothelial (average content of 12.47%) and mesenchymal cells (49.15%) and less hematopoietic cells (23.42%) compared to FiPSC-derived BMOs. Next, the inventors sorted defined cellular subsets using fluorescence-activated cell sorting (FACS) and analyzed their morphology by microscopy. May-Gruenwald-Giemsa staining of cytospins of isolated CD45+CD11b−CD34+ and CD45+CD11b+ and populations showed the typical morphology of myeloid progenitor cells and monocytoid cells, respectively. Sorted BMO-derived CD45−CD31−CD90+CD105+CD271+CD73+ MSPCs were adherent to plastic and possessed typical spindle-like morphology, characteristic of MSPCs. Moreover, these cells expanded in culture and had serial replating capacity for up to 15 passages. Thus, this demonstrates that BMOs consist of endothelial, hematopoietic and mesenchymal cells, contain phenotypically defined HSPCs and MSPCs and can be generated from FiPSCs and UiPSCs.

### Spatial Architecture

The inventors next analyzed the spatial distribution of the distinct cellular subsets by a series of microscopical studies. Confocal imaging revealed spherical shaped CD45+ hematopoietic cells embedded into a network of CD31+ vascular structures and CD271+ stromal cells. To increase the depth of imaging, the inventors used two-photon microscopy, allowing the inventors to capture fluorescent signals of up to 845 μm depth (FIG. 2h). These studies confirmed the presence of vascular, hematopoietic and mesenchymal structures throughout the whole organoid. A dense vascular network is crucial for supporting the hematopoietic niche by providing nutrients and growth factors to HSCs and other niche cells in the BM. The BM niche is composed of different types of blood vessels and thus distinct perivascular domains. Perivascular platelet-derived growth factor beta (PDGFRβ)+ mural cells cover ECs, thereby supporting and promoting vasculogenesis. These PDGFRβ+ pericytes are spatially associated with HSCs and osteoprogenitors and surround arterioles and Type-H vessels in the metaphysis and endosteal regions in mice. Confocal imaging of BMOs followed by 3D surface rendering showed that CD31+ ECs formed a vessel-like network covered by PDGFRβ+ pericytes (FIG. 3). The formation of PDGFR-β+ pericytes changed over time. Whereas on day 10 of differentiation, finger-like extensions of PDGFR-β+ pericytes were only adjacent to ECs, upon full organoid maturation on day 21 PDGFR-β+ pericytes were found to be enwrapping CD31+ cells. This spatial association thus resembles endosteal arterioles and Type-H vessels.

The chemokine CXCL12 signals via the CXCR4 receptor and is the major retention and maintenance factor for HSCs and hematopoietic progenitors in the BM niche32,33. CXCL12-abundant reticular (CAR) cells are characterized by high expression of CXCL12 and their close association to ECs. Moreover, CXCL12 is expressed in ECs themselves. Remarkably, BMOs contained a reticular network of perivascular CXCL12+-cells (FIG. 2e, f) extending protrusions towards the endothelium reminiscent of CAR cells (FIG. 2f). Nestin-expressing perivascular mesenchymal stem cells, known to support and regulate hematopoiesis, are located in endosteal regions in spatial association with HSCs and possess multilineage and self-renewal capacity in mice and human fetal BM. Remarkably, the inventors identified Nestin+ stromal cells in spatial relationship to CD31+ vessels and CD45+ hematopoietic cells in the inventor's BMOs by confocal and two-photon microscopy. Confocal z-stack imaging revealed Nestin+ processes of perivascular mural cells lining the vascular structures as seen in bone marrow in vivo.

When the inventors explored the vessel structure in more detail, the findings indicated that the endothelial and mesenchymal BMO compartments recapitulate key structural features and cell compositions of the human BM niche.

Characterization and Local Distribution of Niche Cells and Hematopoietic Cells within the BMOs as Produced

The present analysis shows that the BMOs consist of a hematopoietic compartment, a vascular compartment, and a stromal (mesenchymal) compartment. Confocal microscopy revealed a self-assembled, interconnected and partially lumen-forming vessel-like network composed of CD31+ endothelial cells covered by a Col IV+ basement membrane and PDGFR-β+ perivascular mesenchymal cells. In addition, flow cytometric analysis showed that the relative composition of endothelial cells and mesenchymal stem cells is comparable to that in native bone marrow (11). Interestingly, the inventors found CXCL12+ cells in a network-like structure closely connected to the endothelial network, reminiscent of the native bone marrow architecture. This strongly suggests the formation of bone marrow-specific CAR cells in the present differentiation system. This is particularly remarkable since CAR cells are the main producers of CXCL12 and SCF, essential signaling molecules for the maintenance and homing of HSCs and the proliferation of lymphoid and erythroid progenitor cells (12). The inventors found that CD45+ hematopoietic cells are distributed in clusters evenly throughout the organoid. This partial clustering could be explained by the increased localization of certain niche cells and their regulatory cytokines in these areas. It is known that hematopoietic stem cells and progenitor cells are localized in the vicinity of blood vessels since growth factors and products produced by niche cells are important regulators for the maintenance of HSCs (11,13).

Differentiation of Myeloid Cells within the BMOs as Produced

In addition, the inventors could show that the present differentiation protocol promotes the autologous differentiation of hematopoietic progenitors into myeloid cells within the BMO structure, indicating a facilitative interaction between hematopoietic progenitors and niche cells. Immunofluorescence showed cell populations positive for markers of monocytes and neutrophils (S100A8/A9, S100A8 and MPO). Interestingly, these cells also had bean-shaped and segmented nuclei, indicating the formation of monocytes and granulocytes at different stages of maturation. Wright-Giemsa staining of a FACS-sorted myeloid population (CD45+CD34−CD11b+) also revealed the emergence of macrophages, monocytes and granulocytes. These results are particularly interesting because the differentiation of HSCs into granulocytes and macrophages depends on GCSF and GM-CSF signaling, but these cytokines are not included in the cytokine cocktail in the present differentiation system. Thus, these results indicate sufficient intrinsic production of these cytokines by niche cells within the BMO system. Although the inventors have not yet been able to demonstrate the emergence of other bloodlines such as lymphocytes, erythrocytes or platelets, CFU assays of FACS-sorted hematopoietic progenitor cells (CD45+CD34+CD11b−) showed the potential of these cells to differentiate into erythrocytes and megakaryocytes. Since the cytokines used in the inventor's system cause a shift in hematopoietic differentiation toward the myeloid cell lineages, future adjustments in cytokine composition could allow differentiation of erythroid or even lymphoid cells.

Neutrophil granulocytes and their precursors make up the largest fraction of nucleated BM cells. The inventors examined whether the BMO niche promotes maturation of blood progenitors into mature cells of the myeloid lineage without the addition of lineage-instructing cytokine, such as G-CSF or GM-SCF.

Organoids were stained for the key myeloid markers S100A8/A937 and myeloperoxidase (MPO). Both S100A8/A9 and MPO expressing cells were found in BMOs, some of them exhibiting a banded or segmented nucleus, indicating myeloid maturation. Immunohistochemical studies confirmed expression of MPO by myeloid cells (FIG. 4c) and TEM revealed cells with electron-dense cytoplasmatic granules, a segmented nucleus and heterochromatin formation at the nuclear margin, which are characteristic features of neutrophil granulocytes. Importantly, the inventors documented orderly maturation of neutrophil granulocytes by flow cytometry: Based on expression of cell surface markers, neutrophil progenitor stages (ProNeu1, ProNeu2, PreNeu), as well as immatureNeus and matureNeus were identified in both FiPSC- and UiPSC-derived BMOs. Light microscopical analysis of flow-sorted and May-Gruenwald-Giemsa-stained cells showed that defined stages of BMO-derived neutrophil granulocytes resemble the morphology of their counterparts in the human BM. Thus, maturation of neutrophil granulocytes within the BMOs, proceeding without addition of recombinant cytokines, is reminiscent of the in vivo situation.

Mesenchymal Stem Cells as Obtained from BMOs Fulfill the Criteria for Mesenchymal Stem Cells.

Mesenchymal stem cells are one of the major cellular components of the bone marrow niche. MSCs maintain bone marrow tissue homeostasis by differentiating into adipocytes, osteocytes, and chondrocytes, while primitive mesenchymal cells and their progeny directly regulate hematopoiesis through the secretion of cytokines (14). In other in vitro bone marrow niche studies, donor primary mesenchymal cells were used to generate the stromal (mesenchymal) compartment of the bone marrow niche, which is then co-cultured with donor hematopoietic cells to mimic the bone marrow hematopoietic compartment. In the present system, the iPSCs instead simultaneously differentiate into hematopoietic cells, endothelial cells, and most notably into mesenchymal progenitor cells such as PDGFRβ+ perivascular cells and CXCL12+ CAR cells. Furthermore, it was demonstrated that the present system induces the formation of functional bona fide mesenchymal stem cells, since BMO-derived MSCs meet the minimum criteria for mesenchymal stem cells proposed by the ISCT: MSCs were negative for the hematopoietic and endothelial surface molecules CD45, CD34, and CD31, but positive for CD90, CD105, CD271 and CD73. The MSCs adhered to plastic and could differentiate into osteoblasts, adipocytes and chondroblasts in vitro (15, 16).

### Modelling Developmental Processes

Having identified arterial pre HE resembling cells by scRNA-seq, the inventors next set out to study endothelial-to-hematopoietic transition (EHT), characterized by morphological changes of arterial ECs and expression of the transcription factor RUNX1. Confocal imaging revealed round RUNX1 expressing cells to be localized in distinct clusters within the organoid. Some of these cells co-expressed CD31 and were still attached to the endothelial wall. Immunofluorescence of immature organoids (day 10) revealed CD31+ cells rounding up from the vessel lining, which was also seen in histological sections of BMOs stained for CD34 and H/E, thus reflecting findings of EHT in vivo. In line with these protein expression data, the inventors found PECAM1+ cells co-expressing RUNX1 in the endothelial cluster of the inventor's scRNA-seq data set.

To capture intermediate states of cellular differentiation in the inventor's flow cytometry data, the inventors analyzed protein expression by unsupervised clustering using the FlowSOM algorithm. The previously assigned endothelial compartment was found to be heterogeneous and contained additional subpopulations. The surface marker expression profile of cells in cluster 3 related to human HE and HSCs, as they were negative for CD45 and co-expressed CD31, CD34, CD90 (Thy1) and CD105 (Endoglin). The adjacent cluster 4 was defined by CD31lowCD34+ and CD45+ cells, constituting the manually gated HSPC cluster. Decreasing CD31 expression with increasing expression of CD34 and CD45 reflects the immunophenotypic sequence of cells that undergo EHT. Remarkably, in the inventor's scRNA-seq dataset the inventors also identified a rare population of cells expressing RUNX1, MECOM, HLF, SPINK2 and MLLT3, signature genes of human fetal HSCs/MPPs. These data suggest that BMOs give rise to hemogenic endothelium, which transition into human HSC/MPP-like cells by EHT. Interestingly, unsupervised clustering also revealed a cluster of cells co-expressing CD271, CD31 and CD105. This marker profile overlaps with endothelium-derived BM stromal cells (eBMSCs) that have been identified in human fetal and regenerative BM. EBMSCs undergo endothelial-to-mesenchymal transition (EndoMT) and show capacity to reconstitute the entire hematopoietic niche after transplantation, including bone progenitors, thereby exhibiting MSC properties. Confocal imaging confirmed the co-expression of CD271, CD31 and CD105 in a subset of ECs, indicative of cells undergoing EndoMT.

### Functional Properties

The phenotypic identification of HSPCs and MSPCs prompted the inventors to investigate their functional properties. To assess the multipotency of BMO-derived MSPCs the inventors performed trilineage differentiation assays. Remarkably, when placed into the respective conditioned medium, isolated MSPCs from both FiPSC- and UiPSC-derived BMOs had the capacity to differentiate into osteogenic, adipogenic and chondrogenic cells, as visualized by Alizarin-Red-staining of calcium deposits, Oil-red-O-staining of lipid vacuoles, and Alcian-blue-staining of glycosaminoglycans, respectively. To examine the multilineage differentiation capacity of BMO-derived HSPCs, the inventors performed colony-forming unit (CFU) assays. FACS-sorted HSPCs gave mainly rise to granulocyte-macrophage progenitor cells (CFU-GM), but also multipotential granulocyte, erythroid, macrophage and megakaryocyte progenitor cells (CFU-GEMM) and erythroid progenitors (BFU-E). Isolated HSPCs from UiPSC-derived BMOs also gave rise to CFU-GEMM and CFU-GM, yet not to typical BFU-E colonies. Colony identity was validated by May-Gruenwald-Giemsa staining of picked colonies. These findings demonstrate that BMOs contain multipotent MSPCs and HSPCs. In order to determine functional properties of BMOs in vivo, the inventors transplanted the BMOs on day 21 of differentiation under the renal capsule of immunodeficient NOD/SCID/IL2Rγnull (NSG) mice. Analysis at 3.5 months after transplantation revealed further growth of organoids. Histological analysis of organoid sections after transplantation displayed blood vessels filled with erythrocytes. Strikingly, the inventors found human CD45+ cells in the BM of transplanted mic. These data indicate that the organoids gain access to the mice vasculature and that BMO-derived human CD45+ cell home to the murine BM.

### Disease Modelling

In order to test the suitability of the produced BMOs for modeling bone marrow-related diseases, the inventors generated VPS45-deficient BMOs in a proof-of-concept approach. Genetic VPS45 deficiency in patients results in disease associated with severe congenital neutropenia and myelofibrosis (17). Although there were no striking differences in the relative composition of hematopoietic cells and niche cells between WT and VPS45 BMOs, preliminary histological results of VPS45 BMOs indicated an increased formation of reticulin fibers as a sign of myelofibrosis in the VPS45-mutated BMOs.

Finally, the inventors tested whether the inventor's novel BMOs can indeed be used as a model system to recapitulate the phenotype of monogenic BM diseases. Children with Vacuolar Protein Sorting 45 Homolog (VPS45) deficiency present with neutropenia and myelofibrosis in the first year of life, progressing to BM failure. Their BM is hypercellular and shows myeloid hyperplasia associated with increased apoptosis and functional deficiencies of neutrophils. The inventors generated BMOs from a gene-edited iPSC derivative line with the homozygous Thr224Asn mutation in VPS4514 and compared them to isogenic control BMOs (wild type, WT). No major differences in the composition of hematopoietic cells and niche cells were seen. However, VPS45-deficient BMOs showed increased deposition of reticulin fibers, reminiscent of myelofibrosis in BM biopsies of VPS45 deficient patients. This was accompanied by expansion of alpha smooth muscle actin (SMA) expressing myofibroblast-like stromal cells, previously described as critical drivers of myelofibrosis.

Flow cytometric analysis of VPS45-deficient BMOs showed higher numbers of matureNeus in comparison to controls resembling myeloid hyperplasia and a significant increase in AnnexinV expression on VPS45-mutant matureNeus, indicating enhanced apoptosis of this subpopulation, a feature also seen in patients. Thus, BMOs may provide novel model systems for dissecting genes and pathways in previously intractable pathomechanisms of BM failure diseases.

### LITERATURE AS CITED

1. Pinho S, Frenette P S. Haematopoietic stem cell activity and interactions with the niche. Nat Rev Mol Cell Biol. 2019 May 1; 20(5):303-20.
2. Asada N, Takeishi S, Frenette P S. Complexity of bone marrow hematopoietic stem cell niche. Int J Hematol. 2017 Jul. 1; 106(1):45-54.
3. Raaijmakers MHGP, Mukherjee S, Guo S, Zhang S, Kobayashi T, Schoonmaker J A, et al. Bone progenitor dysfunction induces myelodysplasia and secondary leukaemia. Nature. 2010 April; 464(7290):852-7.
4. Giger S, Hofer M, Miljkovic-Licina M, Hoehnel S, Brandenberg N, Guiet R, et al. Microarrayed human bone marrow organoids for modeling blood stem cell dynamics [Internet]. Bioengineering; 2021 May [cited 2021 Aug. 28]. Available from: http://biorxiv.org/lookup/doi/10.1101/2021.05.26.445803
5. Chou D B, Frismantas V, Milton Y, David R, Pop-Damkov P, Ferguson D, et al. On-chip recapitulation of clinical bone marrow toxicities and patient-specific pathophysiology. Nat Biomed Eng. 2020 April; 4(4):394-406.
6. Isern J, Martin-Antonio B, Ghazanfari R, Martin A M, López JA, del Toro R, et al. Self-renewing human bone marrow mesenspheres promote hematopoietic stem cell expansion. Cell Rep. 2013 May 30; 3(5):1714-24.
7. Li M, Izpisua Belmonte J C. Organoids Preclinical Models of Human Disease. N Engl J Med. 2019 Feb. 7; 380(6):569-79.
8. Niwa A, Heike T, Umeda K, Oshima K, Kato I, Sakai H, et al. A Novel Serum-Free Monolayer Culture for Orderly Hematopoietic Differentiation of Human Pluripotent Cells via Mesodermal Progenitors. PLOS ONE. 2011 Jul. 27; 6(7):e22261.
9. Wimmer R A, Leopoldi A, Aichinger M, Kerjaschki D, Penninger J M. Generation of blood vessel organoids from human pluripotent stem cells. Nat Protoc. 2019 Nov. 1; 14(11):3082-100.
10. Wimmer R A, Leopoldi A, Aichinger M, Wick N, Hantusch B, Novatchkova M, et al. Human blood vessel organoids as a model of diabetic vasculopathy. Nature. 2019 Jan. 1; 565(7740):505-10.
11. Crane G M, Jeffery E, Morrison S J. Adult haematopoietic stem cell niches. Nat Rev Immunol. 2017 September; 17(9):573-90.
12. Nagasawa T, Omatsu Y, Sugiyama T. Control of hematopoietic stem cells by the bone marrow stromal niche: the role of reticular cells. Trends Immunol. 2011 July; 32(7):315-20.
13. Kokkaliaris K D, Kunz L, Cabezas-Wallscheid N, Christodoulou C, Renders S, Camargo F, et al. Adult blood stem cell localization reflects the abundance of reported bone marrow niche cell types and their combinations. Blood. 2020 Nov. 12; 136(20):2296-307.
14. Kfoury Y, Scadden D T. Mesenchymal cell contributions to the stem cell niche. Cell Stem Cell. 2015 Mar. 5; 16(3):239-53.
15. Horwitz E M, Le Blanc K, Dominici M, Mueller I, Slaper-Cortenbach I, Marini F C, et al. Clarification of the nomenclature for MSC: The International Society for Cellular Therapy position statement. Cytotherapy. 2005; 7(5):393-5.
16. Dominici M, Le Blanc K, Mueller I, Slaper-Cortenbach I, Marini F, Krause D, et al. Minimal criteria for defining multipotent mesenchymal stromal cells. The International Society for Cellular Therapy position statement. Cytotherapy. 2006; 8(4):315-7.
17. Vilboux T, Lev A, Malicdan M C, Simon A J, Jarvinen P, Racek T, et al. A congenital neutrophil defect syndrome associated with mutations in VPS45. N Engl J Med. 2013 Jul. 4; 369(1):54-65.

1-21. (canceled)

22. A method for producing mammalian bone marrow organoids, comprising:
a) generating aggregates from pluripotent stem cells (PSCs) in an aggregation medium comprising at least one Rho-associated protein kinase (ROCK)-inhibitor,
b) inducing mesoderm in said aggregates of step a), comprising culturing said aggregates in a serum-free culture medium supplemented with one or more of Bone Morphogenetic Protein 4 (BMP4), Glycogen Synthase Kinase (GSK) 3 inhibitor, and Vascular Endothelial Growth Factor (VEGF),
c) replacing the medium of the culture of b) with a medium supplemented with one or more of VEGF, fibroblast growth factor (FGF)-2, stem cell factor (SCF), and an inhibitor of TGF-β/Activin/Nodal pathway, and
d) inducing hematopoiesis within the mesoderm-induced aggregates in a 3D suspension matrix cytokine induction system or embedding the mesoderm-induced aggregates into a suitable polymerized 3 D matrix;
whereby the mammalian bone marrow organoids are produced.

23. The method for producing mammalian bone marrow organoids according to claim 22, wherein the 3D polymerized matrix comprises an extracellular matrix protein.

24. The method for producing mammalian bone marrow organoids according to claim 23 wherein the 3D polymerized matrix comprises collagen and Matrigel® and wherein the collagen and Matrigel® are at a ratio of collagen/Matrigel® of between about 1:1 and 4:1.

25. The method for producing mammalian bone marrow organoids according to claim 22, wherein the inducing step d) further comprises culturing the 3D suspension aggregates or embedding the mesoderm-induced aggregates in a medium supplemented with one or more of VEGF, FGF-2, SCF, and SB431542.

26. The method for producing mammalian bone marrow organoids according to claim 22, wherein no lineage directing cytokines selected from the group consisting of EPO, IL-6 and G-CSF are used in any of the steps.

27. The method for producing mammalian bone marrow organoids according to claim 22, the method further comprising extracting individual vascular networks generated in step d) followed by culturing in a medium supplemented with VEGF, SCF, IL-3, Flt-3L and TPO.

28. The method for producing mammalian bone marrow organoids according to claim 27, further comprising the step of:
(i) fixing the vascular networks in step e) and performing immunofluorescence analysis and/or dissociating the vascular networks into individual cells and performing flow cytometry analysis and/or live-cell imaging; and/or
(ii) fixing the organoids as produced and performing immunofluorescence analysis and/or dissociating the organoids into individual cells and performing flow cytometry analysis and/or live-cell imaging.

29. The method for producing mammalian bone marrow organoids according to claim 22, further comprising the step of culturing the organoids for a maximum of about 60 days.

30. The method for producing mammalian bone marrow organoids according to claim 22, wherein the PSCs are provided by dissociating PSC cells into a single cell suspension.

31. The method for producing mammalian bone marrow organoids according to claim 22, wherein the ROCK-inhibitor is selected from the group consisting of Y-27632 ((1R,4r)-4-((R)-1-aminoethyl)-N-(pyridin-4-yl)cyclohexanecarboxamide) and fasudil.

32. The method for producing mammalian bone marrow organoids according to claim 22, wherein the aggregation medium comprises one or more of a basal medium, a serum substitute, L-glutamine and essential amino acids.

33. The method for producing mammalian bone marrow organoids according to claim 22, wherein the serum-free cell culture medium is a TeSR® medium.

34. The method for producing mammalian bone marrow organoids according to claim 22, wherein the glycogen synthase kinase (GSK) 3 inhibitor is CHIR99021.

35. The method for producing mammalian bone marrow organoids according to claim 22, wherein the pluripotent stem cells are induced pluripotent stem cells or embryonic stem cells.

36. The method for producing mammalian bone marrow organoids according to claim 35, wherein the pluripotent stem cells are isolated from a mammal selected from the group consisting of a human, a mouse, a monkey, a rat, a pig, a dog, a cat, a rabbit, a sheep, cattle, an equine, and a goat.

37. A vascular network or mature mammalian bone marrow organoid, produced according to the method of claim 22, or a pharmaceutical composition comprising the vascular network and/or mature mammalian bone marrow organoid.

38. An assembloid comprising the vascular network or mature mammalian bone marrow organoid according to claim 37 and at least one additional organoid.

39. The assembloid according to claim 38, further comprising immune cells, blood vessels, and pericytes.

40. A method, wherein the method comprises using the vascular network or mature mammalian bone marrow organoid according to claim 37, or an assembloid comprising the vascular network or mature mammalian bone marrow organoid according to claim 37 and at least one additional organoid:
(i) as a model system for a bone marrow related disease, such as, a hematological disease, severe congenital neutropenia, myelofibrosis, blood cell cancers, anemia, thrombocytopenia, inborn errors of hematopoiesis and immunity, conditions related to HIV, sickle cell disease, and complications from chemotherapy or transfusions; and/or
(ii) as a model system in the pathogenesis of a bone marrow related disease, such as, a hematological disease, severe congenital neutropenia, myelofibrosis, blood cell cancers, anemia, thrombocytopenia, inborn errors of hematopoiesis and immunity, conditions related to HIV, sickle cell disease, and complications from chemotherapy or transfusions; and/or
(iii) as a model system for identifying and/or testing pharmaceutically effective compounds for treating or preventing of a bone marrow related disease, such as, a hematological disease, severe congenital neutropenia, myelofibrosis, blood cell cancers, anemia, thrombocytopenia, inborn errors of hematopoiesis and immunity, conditions related to HIV, sickle cell disease, and complications from chemotherapy or transfusions; and/or
(iv) for the in-vitro production of BMOs or mammalian blood cells, in particular autologous BMOs or mammalian blood cells, in particular for transplantation.

41. A method for the treatment of a bone marrow related disease, a condition related to HIV, sickle cell disease, or a complication from chemotherapy or transfusion, wherein the method comprises administering, to a subject in need of such treatment, a pharmaceutically effective amount of the vascular network or mature mammalian bone marrow organoid according to claim 37, or an assembloid comprising the vascular network or mature mammalian bone marrow organoid according to claim 37 and at least one additional organoid.
